(19) United States
(12) Patent Application Publication
(10) Pub. No.: US 2010/0105031 A1
(43) Pub. Date: Apr. 29, 2010

(54) METHOD FOR PREDICTION OF THE EFFICACY OF VASCULARIZATION INHIBITOR

(75) Inventors: Junji Matsui, Ibaraki (JP); Taro Semba, Ibaraki (JP)

Correspondence Address: DICKSTEIN SHAPIRO LLP, 1633 Broadway, NEW YORK, NY, 10019, US

(73) Assignee: Esai R & D Management Co., Ltd., Tokyo (JP)

(21) Appl. No.: 11/997,543

(22) PCT Filed: Aug. 1, 2006
(86) PCT No.: PCT/JP2006/315563

§ 371 Date: Jan. 31, 2008

(30) Foreign Application Priority Data
Aug. 1, 2005 (JP) 2005-223440

(51) Int. Cl. C12Q 1/68; C12Q 1/02; G01N 33/53

(52) U.S. Cl. 435/6; 435/29; 435/7.1

(57) ABSTRACT

Disclosed is a method for the prediction of the efficacy of a vascularization inhibitor. In the method, the anti-tumor effect of a vascularization inhibitor can be predicted by measuring the number of blood vessels surrounded by pericytes in a tumor and using the measurement value as a measure for the anti-tumor effect.

### TECHNICAL FIELD

The present invention relates to a novel method for predicting the effect of angiogenesis inhibitors (Vascularization Inhibitors), such as substances having vascular endothelial growth factor (hereinafter, sometimes referred to as “VEGF”) inhibitory activity (hereinafter, sometimes referred to as “VEGF inhibitors”).

### BACKGROUND ART

Clinical trials have made it clear that angiogenesis inhibitors are useful as antitumor agents. For example, bevacizumab that is a neutralizing antibody against VEGF playing an important role among angiogenic processes is reported to have shown an antitumor effect against colorectal cancer in clinical trials⁽¹⁾.

4-(3-chloro-4-(cyclopropylaminocarbonyl)-aminophenoxy)-7-methoxy-6-quinolinecarboxamide is reported as an angiogenesis inhibitor^{(2 and 3)}.

Evaluating the effect of angiogenesis inhibitors, determining the effective dose of angiogenesis inhibitors and predicting the effect of angiogenesis inhibitors prior to administration thereof are very useful for efficiently performing treatment with angiogenesis inhibitors and for contributing to the improvement of patients' QOL⁽⁴⁾. With respect to the former two matters, a great number of researches are now being carried out⁽⁵⁾. Specifically, methods such as dynamic contrast-enhanced magnetic resonance imaging (DCE-MRI), positron emission tomograpy (PET), interstitial fluid pressure and serum VEGF are reported. Among all, DCE-MRI is believed to be effective as a method for evaluating the effect of angiogenesis inhibitors⁽⁶⁾.

On the other hand, predicting the effect of angiogenesis inhibitors prior to administration thereof is very beneficial and important to patients for avoiding the administration of inefficient medicine and reducing adverse effect⁽⁴⁾. However, no effective method for predicting the effect of angiogenesis inhibitors prior to administration thereof has been found yet.

### REFERENCES

(1) Bevacizumab plus irinotecan, fluorouracil, and leucovorin for metastatic colorectal cancer, N Engl J Med. 2004, 350, 2335-42
(2) WO 02/32872
(3) WO 2004/080462
(4) Inhibition of vascular endothelial growth factor (VEGF) signaling in cancer causes loss of endothelial fenestrations, regression of tumor vessels, and appearance of basement membrane ghosts. Am J Pathol., 2004, 165, 35-52
(5) Direct evidence that the VEGF-specific antibody bevacizumab has antivascular effects in human rectal cancer, Nature Medicine, 2004, 10, 145-147
(6) Dynamic contrast-enhanced magnetic resonance imaging as a biomarker for the pharmacological response of PTK787/ZK 222584, an inhibitor of the vascular endothelial growth factor receptor tyrosine kinases, in patients with advanced colorectal cancer and liver metastases: results from two phase I studies., J Clin Oncol., 2003, 21, 3955-64.

### DISCLOSURE OF THE INVENTION

Under such circumstances, the present invention has been made. It is an object of the invention to find a method for predicting the effect of angiogenesis inhibitors. As a result of extensive and intensive researches toward the solution of the above problem, the present inventors have found out for the first time that the antitumor effect of angiogenesis inhibitors correlates with the number of those blood vessels which are coated with pericytes in the relevant tumor. The present inventors have also found out that it is possible to predict the antitumor effect of angiogenesis inhibitors by determining the number of those blood vessels which are covered with pericytes in the relevant tumor and using the resultant number as an indicator.

The present invention relates to the following.

(1) A method of predicting the antitumor effect of an angiogenesis inhibitor, comprising the following steps:

a step of determining the ratio of those blood vessels which are covered with pericytes in a tumor; and

a step of judging whether or not a cancer patient is highly sensitive to the angiogenesis inhibitors by using as an indicator the resultant ratio of those blood vessels which are covered with pericytes.

The method described in (1) above may further comprise a step of determining the number of blood vessels in the tumor; and a step of judging whether or not the cancer patient is highly sensitive to the angiogenesis inhibitort by using as an indicator the ratio of the number of those blood vessels which are covered with pericytes in the tumor to the number of blood vessels in the tumor.

(2) A method of predicting the antitumor effect of an angiogenesis inhibitor, comprising the following steps:

a step of determining the number of blood vessels in a tumor and the number of those blood vessels which are covered with pericytes in the tumor; and

a step of judging whether or not a cancer patient is highly sensitive to the angiogenesis inhibitor by using as an indicator the ratio of the number of those blood vessels which are covered with pericytes in the tumor to the number of blood vessels in the tumor.

In the method described in (1) or (2) above, the tumor may be a tumor collected from the cancer patient.

In the method described in (1) or (2) above, the determination of the number of those blood vessels which are covered with pericytes may be performed by using as an indicator the expression of at least one substance selected from the group consisting of α-SMA, desmin, chondroitin sulfate proteoglycan 4, calponin, caldesmon and PDGF receptor. Among all, the expression of α-SMA and/or desmin is used preferably as an indicator. The determination of the number of those blood vessels which are covered with pericytes may be performed by, for example, an immunochemical method, in situ hybridization or quantitative RT-PCR.

Further, in the method described in (1) or (2) above, the determination of the number of blood vessels in the tumor may be performed by using as an indicator the expression of at least one substance selected from the group consisting of CD31, wVF, CD34, CD105, CXCR4, CD146, CD133, KDR and KIT. Among all, the expression of CD31 is used preferably as an indicator. The determination of the number of blood vessels in the tumor may be performed by, for example, an immunochemical method, in situ hybridization or quantitative RT-PCR.

In the method described in (1) or (2) above, the angiogenesis inhibitor is, for example, a VEGF receptor kinase inhibitor. Examples of VEGF receptor kinase inhibitors may be given as follows.

A compound represented by the following general formula (I), a pharmacologically acceptable salt thereof, or a solvate of the compound or the salt:

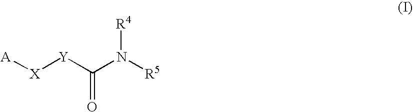

wherein A is a group represented by one of the following formulas:

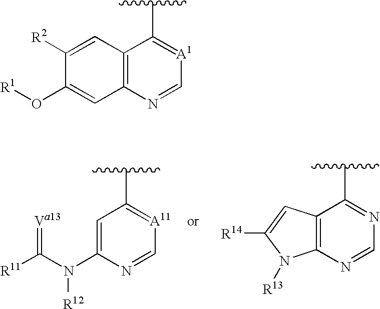

(wherein R¹ is a group represented by a formula —V¹—V²—V³ (where V¹ is a C₁₋₆ alkylene group which may have a substituent(s); V² is a single bond, an oxygen atom, a sulfur atom, a carbonyl group, a sulfinyl group, a sulfonyl group, a group represented by a formula —CONR⁶—, a group represented by a formula —SO₂NR⁶—, a group represented by a formula —NR⁶SO₂—, a group represented by a formula —NR⁶CO— or a group represented by a formula —NR⁶— (where R⁶ is a hydrogen atom, a C₁₋₆ alkyl group which may have a substituent(s) or a C₃₋₈ cycloalkyl group which may have a substituent(s)); and V³ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s));
R² is a cyano group, a C₁₋₆ alkoxy group which may have a substituent(s), a carboxyl group, a C₂₋₂₇ alkoxycarbonyl group which may have a substituent(s) or a group represented by a formula —CONV^{a11}V^{a12} (where V^{a11} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s); and V^{a12} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s), a hydroxyl group, a C₁₋₆ alkoxy group which may have a substituent(s) or a C₃₋₈ cycloalkoxy group which may have a substituent(s));
A¹ is a carbon atom or a nitrogen atom which may have a substituent(s);
R¹¹ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s) or a mono-C₁₋₆ alkylamino group which may have a substituent(s);
R¹² is a hydrogen atom or a C₁₋₆ alkyl group which may have a substituent(s);
V^{a13} is an oxygen atom or a sulfur atom;
A¹¹ is a carbon atom or a nitrogen atom which may have a substituent(s);
R¹³ is a hydrogen atom, a C₁₋₆ alkyl group which may have a substituent(s) or a C₃₋₈ cycloalkyl group which may have a substituent(s);
R¹⁴ is a group represented by a formula —V^{a14}—V^{a15} (where V^{a14} is a single bond or a carbonyl group; and V^{a15} is a hydrogen atom, a hydroxyl group, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s), an amino group, a mono-C₁₋₆ alkylamino group which may have a substituent(s), a di-C₁₋₆ alkylamino group which may have a substituent(s), a formyl group, a carboxyl group or a C₂₋₇ alkoxycarbonyl group which may have a substituent(s));
X is an oxygen atom or a sulfur atom;
Y is a group represented by one of the following formulas:

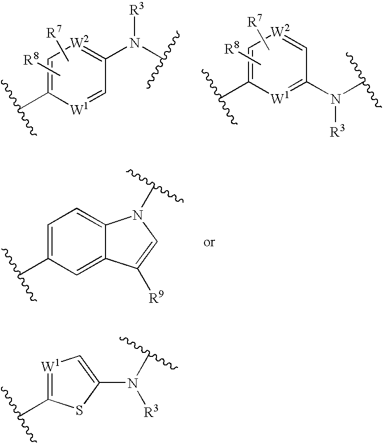

(wherein R³ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₂₋₇ acyl group which may have a substituent(s) or a C₂₋₇ alkoxycarbonyl group which may have a substituent(s);
R⁷ and R⁸ independently of each other represent a hydrogen atom, a halogen atom, a cyano group, a nitro group, an amino group, a C₁₋₆ alkyl group which may have a substituent(s), a C₃₋₈ cycloalkyl group which may have a substituent(s), a C₁₋₆ alkoxy group which may have a substituent(s), a C₁₋₆ alkylthio group which may have a substituent(s), a formyl group, a C₂₋₇ acyl group which may have a substituent(s), a C₂₋₇ alkoxycarbonyl group which may have a substituent(s) or a group represented by a formula —CONV^{d1}V^{d2} (where V^{d1} and V^{d2} independently of each other represent a hydrogen atom or a C₁₋₆ alkyl group which may have a substituent(s));
R⁹ is a hydrogen atom, a halogen atom or a C₁₋₆ alkyl group which may have a substituent(s); and
W¹ and W² independently of each other represent a carbon atom or a nitrogen atom which may have a substituent(s));
R⁴ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₂₋₇ acyl group which may have a substituent(s) or a C₂₋₇ alkoxycarbonyl group which may have a substituent(s); and
R⁵ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s).

In the method described in (1) or (2) above, the VEGF receptor kinase inhibitor is preferably 4-(3-chloro-4-(cyclopropylaminocarbonyl)aminophenoxy)-7-methoxy-6-quinolinecarboxamide, a pharmacologically acceptable salt thereof, or a solvate of the compound or the salt.

Further, in the method described in (1) or (2) above, for example, the angiogenesis inhibitor may be at least one substance selected from the group consisting of anti-VEGF receptor antibody, anti-VEGF antibody, FGF receptor kinase inhibitor, PDGF receptor kinase inhibitor, EGF receptor kinase inhibitor, anti-FGF receptor antibody, anti-PDGF receptor antibody, anti-EGF receptor antibody, anti-FGF antibody, anti-PDGF antibody and anti-EGF antibody.

(3) A kit for use in the method described in (1) or (2) above, comprising at least one antibody selected from the group consisting of anti-α-SMA antibody, anti-desmin antibody, anti-chondroitin sulfate proteoglycan 4 antibody, anti-calponin antibody, anti-caldesmon antibody and anti-PDGF receptor antibody.

(4) A kit for use in the method described in (1) or (2) above, comprising a polynucleotide comprising a sequence complementary to at least a part of a transcript RNA from at least one gene selected from the group consisting of α-SMA gene, desmin gene, chondroitin sulfate proteoglycan 4 gene, calponin gene, caldesmon gene and PDGF receptor gene.

According to the present invention, a method of predicting the antitumor effect of an angiogenesis inhibitor is provided.

More specifically, it has become possible to predict the antitumor effect of an angiogenesis inhibitor by determining the number of those blood vessels which are covered with pericytes in a tumor and using the resultant number as an indicator.

Since the method according to the present invention enables to predict the antitumor effect of an angiogenesis inhibitor without administering the agent to patients, it has become possible to select and treat those patients who are expected to show higher antitumor effect. Thus, contribution to patients' QOL has become possible.

### BRIEF DESCRIPTION OF THE DRAWINGS

FIG. 1 shows the correlation between the antitumor effect of 4-(3-chloro-4-(cyclopropylaminocarbonyl)aminophenoxy)-7-methoxy-6-quinolinecarboxamide and the number of those blood vessels which are covered with pericytes in tumor tissue in human cancer cell strain transplanted mouse models.

FIG. 2 shows the correlation between the antitumor effect of angiogenesis inhibitors and the number of those blood vessels which are covered with pericytes in human cancer cell lines transplanted mouse models. In FIG. 2, Compound 1 represents 4-(3-chloro-4-(cyclopropylaminocarbonyl)aminophenoxy)-7-methoxy-6-quinolinecarboxamide and Compound 2 represents 5-(5-fluoro-2-oxo-1,2-dihydroindole-3-ylidenemethyl)-2,4-dimethyl-1H-pyrrole-3-carboxylic acid (2-diethylaminoethyl)amide.

FIG. 3 shows the correlation between the antitumor effect of 4-(3-chloro-4-(cyclopropylaminocarbonyl)aminophenoxy)-7-methoxy-6-quinolinecarboxamide and a pericyte marker desmin in tumor tissue in human cancer cell lines subcutaneously transplanted mouse models.

### BEST MODE FOR CARRYING OUT THE INVENTION

Hereinbelow, the embodiments of the present invention will be described. The following embodiments are provided only to illustrate the present invention, and they are not intended to limit the present invention only to these embodiments. The present invention may be carried out in various embodiments without departure of the spirit of the present invention.

All publications, patents and other patent documents cited herein are incorporated herein by reference in their entirety. The contents of the scope of claim, specification, drawings and abstract of Japanese Patent Application No. 2005-223440 filed on Aug. 1, 2005 based on which the present patent application claims priority are incorporated herein by reference in their entirety.

The present invention provides a method of prediction the antitumor effect of an angiogenesis inhibitor, comprising a step of determining the number of those blood vessels which are covered with pericytes in a tumor; and a step of judging whether or not a cancer patient is highly sensitive to the angiogenesis inhibitor by using as an indicator the resultant number of those blood vessels which are covered with pericytes.

1. Step of Determining the Number of Those Blood Vessels which are Covered with Pericytes in Tumor

In this step, the tumor is preferably a tumor removed from the cancer patient. Such a tumor may be obtained by removing a tumor tissue from the cancer patient by surgical treatment (e.g., biopsy).

The size of tumor sample to be removed from cancer patients is not particularly limited. Any size may be used as long as the tumor sample allows determination of the number of those blood vessels covered with pericytes therein. For example, if the tumor is a solid cancer, the size of tumor sample to be removed may be a size of a tumor sample taken by biopsy (e.g., 2-3 mm) or a size of a tissue section removed with a surgical knife (e.g., the size of grain of rice).

The type of tumor used in the present invention is not particularly limited. For example, brain tumor, head&neck cancer, esophageal cancer, tongue cancer, lung cancer, breast cancer, pancreatic cancer, gastric cancer, cancer of the small intestine or duodenum, large bowel cancer (colon cancer, rectal cancer), bladder cancer, renal cancer, liver cancer, prostate cancer, uterine cancer, ovary cancer, thyroid cancer, gallbladder cancer, pharyngeal cancer, sarcoma (e.g., osteosarcoma, chondrosarcoma, Kaposi sarcoma, myosarcoma, angiosarcoma, fibrosarcoma or the like), leukemia (e.g., chronic myelogenous leukemia (CML), acute myelogenous leukemia (AML), chronic lymphocytic leukemia (CLL), acute lymphocytic leukemia (ALL), lymphoma, malignant lymphoma, multiple myeloma (MM) or the like), melanoma and so forth may be enumerated.

Pericytes exists surrounding blood capillaries and veins, and they are referred to perithelial cells.

Pericytes express α-smooth muscle actin (hereinafter, sometimes referred to as “α-SMA”), desmin, chondroitin sulfate proteoglycan 4 (hereinafter, sometimes referred to as “NG-2”), calponin, caldesmon (Characterization of smooth muscle cell and pericyte differentiation in the rat retina in vivo, Investigative. Ophthalmology. Visual Science. 45, 2795-2806, 2004), platelet-derived growth factor receptor (hereinafter, sometimes referred to as “PDGF receptor”) (Cellular abnormalities of blood vessels as targets in cancer, Current Opinion in Genetics and Development, 15, 102-111. 2005), etc. and do not express factor VIII (A new method for isolation of smooth muscle cells from human umbilical cord arteries, Scand J. Clin. Lab. Invest. 57, 21-29, 1997) and GFAP (Localization of Brain Endothelial Luminal and Abluminal Transporters with Immunogold Electron Microscopy, NeuroRx., 2, 27-43, 2005). Therefore, pericytes may be distinguished from other cells by examining the presence or absence of the expression of these substances.

The term “blood vessels (which are) covered with pericytes” means blood vessels which are totally or partially surrounded by pericytes.

The “number of blood vessels (which are) covered with pericytes” may be calculated, for example, as the number of blood vessels covered with pericytes per unit area in a tumor, or as the number of blood vessels covered with pericytes per unit volume in a tumor, or as the number of blood vessels covered with pericytes per unit weight.

In this step, the number of blood vessels covered with pericytes may be determined, for example, by using as an indicator the expression of a protein and/or mRNA which is expressed in pericytes.

Examples of proteins and/or genes expressed in pericytes include α-SMA, desmin, chondroitin sulfate proteoglycan 4, calponin, caldesmon and PDGF receptor. Preferably, α-SMA or desmin is used. For example, by measuring the expression of these proteins and/or mRNAs in tumor samples collected from patients, it is possible to obtain information (such as the types of proteins and/or genes expressed in the tumor samples, the presence or absence of expression thereof, or the expression levels thereof). Using this information as an indicator, it is possible to calculate the number of those blood vessels covered with pericytes.

Measurement of protein may be performed by such methods as immunochemical methods (e.g., immunohistochemical methods or Western blotting) or mass spectrometry. Preferably, immunochemical methods are used. Particularly preferably, immunohistochemical methods are used. These methods may be performed according to conventional procedures.

On the other hand, measurement of mRNA may be performed by such methods as in situ hybridization, Northern blotting, DNA microarray, RT-PCR and quantitative RT-PCR. Preferably, in situ hybridization and quantitative RT-PCR may be enumerated. These methods may be performed according to conventional procedures.

In situ hybridization may be performed, for example, according to the method described in “Jikkenn Igaku Bessatu, Shin-Idenishikogalcu Handbook” (Experimental Medicine Special Issue, New Genetic Engineering Handbook), Chapter 4, published by Yodosha in 2003.

Hereinbelow, one example of a method of determining the number of blood vessels covered with pericytes will be described.

The number of blood vessels covered with pericytes may be determined by an immunohistochemical method using as an indicator the expression of a protein(s) expressed specifically in pericytes.

The immunohistochemical method may be performed according to conventional procedures (“Saibo-Kogaku Bessatu, Me de Mini jikkenn note series, Bio-Jikkenn Illustrated Vol. 5, Tanpaku-nante-Kowakunai” (Special Issue of Cell Engineering, Visual Experimental Note Series, Illustrated Biological Experiments, Vol. 5 “Who's Afraid of Proteins”), Chapter 5, Immunostaining, pp. 127-163, published by Shujunsha Co., Ltd., 1997).

First, tissue sections are prepared from tumor samples removed from cancer patients. Examples of tissue sections include frozen sections and paraffin sections.

Tumor samples removed from patients may be either untreated or treated for fixation. The tumor samples may be embedded with OCT compound or the like.

Fixation treatment may be performed with formaldehyde, preferably 4% PFA/PBS(−). Then, the formaldehyde may be replaced with 20% sucrose/phosphate buffer or the like.

Various conditions for these operations may be selected appropriately depending on the protein to be measured and the antibody to be used.

The tissue section may be retained on a slide glass and pretreated to make staining possible. The method of this pretreatment is not particularly limited and may be appropriately selected depending on the protein to be measured and the antibody to be used. For example, the tissue section may be pretreated with a solution containing xylene, formaldehyde, acetone, methanol, etc. Alternatively, the tissue section may be pretreated with a solution containing BSA, Triton-X100, tween 20, skim milk, casein, etc.

Subsequently, the pretreated tissue section is contacted with an antibody that recognizes a protein to be measured (hereinafter, sometimes referred to as the “primary antibody”). The primary antibody may be a commercially available antibody or may be prepared. The primary antibody may be labeled with a labeling agent or may not be labeled. When the primary antibody is not labeled, an antibody that recognizes the primary antibody (hereinafter, sometimes referred to as the “secondary antibody”) may be contacted therewith. The secondary antibody is preferably labeled with a labeling agent. Examples of the labeling agent include enzymes (such as alkaline phosphatase, peroxidase, glucose oxidase, (β-galactosidase), fluorescent substances (such as FITC (fluorescein isothiocyanate), Alexa488, PE, Rhodamin, Texas Red, Cy3, Cy5, allophycocyanin, PharRed, DsRed, AmCyan, ZsGreen, ZsYellow, AsRed, HcRed) and biotin. When the labeling agent is biotin, avidin or streptavidin may be further contacted. Such avidin or streptavidin is preferably labeled with a labeling agent. Examples of the labeling agent include enzymes (such as alkaline phosphatase, peroxidase, glucose oxidase, (β-galactosidase) and fluorescent substances (such as FITC, Alexa488, PE, Rhodamin, Texas Red, Cy3, Cy5, allophycocyanin, PharRed, DsRed, AmCyan, ZsGreen, ZsYellow, AsRed, HcRed). Various conditions of reactions (such as reaction solution, antibody concentration, reaction time, reaction temperature, washing procedure, etc.) may be appropriately selected depending on the protein to be measured and the antibody to be used.

When the labeling agent is an enzyme, a substrate and/or a coloring reagent is contacted with the tissue section for coloring. By observing this coloring, it is possible to determine the number of blood vessels coated with pericytes.

When the enzyme is peroxidase, a substrate such as H₂O₂ and a coloring reagent such as diaminobenzidine (DAB) may be contacted with the tissue section.

When the enzyme is alkaline phosphatase, a substrate such as 5-bromo-4-chloro-3-indolyl phosphate and a coloring reagent such as nitrobluetetrazorium may be contacted with the tissue section. When the enzyme is alkaline phosphatase, it is also possible to perform a chemiluminescent reaction by contacting a coloring substrate such as CSPD (disodium 3-(4-methoxyspiro{1,2-dioxetane-3,2′-(5′-chloro)tricyclo[3.3.1.1^{3,7}]-decan}-4-yl)phenylphosphate) with the tissue section.

When the labeling agent is a fluorescent substance, the number of blood vessels coated with pericytes may be measured by irradiating the tissue section with excitation light for luminescence and observing the resultant fluorescence.

Further, the treated tissue section may be nuclear stained with hematoxylin or methyl green.

Further, the treated tissue section may be mounted with an aqueous mounting medium.

Thus, the number of blood vessels covered with pericytes per unit area in tumor may be calculated. Alternatively, the number of blood vessels covered with pericytes may be calculated as a value per unit volume of the tumor or as a value per unit weight of the tumor.

Hereinbelow, another example of the method of determining the number of blood vessels covered with pericytes will be described.

It is possible to determine the number of blood vessels covered with pericytes by quantitative RT-PCR using as an indicator the expression of an mRNA expressed specifically in pericytes.

First, RNA is purified from a tumor sample removed from a cancer patient.

TRIZOL reagent (Invitrogen) is added to the tumor sample to homogenize the tumor tissue. Subsequently, chloroform is added to the homogenized tumor. The resultant solution is shook and agitated vigorously for 15 sec, left at room temperature for 2 to 3 min, and then centrifuged (12,000×g, 10 min, 4° C.). After centrifugation, the aqueous layer is transferred to a fresh tube. To this tube, isopropyl alcohol is added. After leaving at room temperature for 10 min, the tube was centrifuged (12,000×g, 10 min, 4° C.). The resultant precipitate is washed with 75% ethanol to thereby purify RNA.

Quantitative RT-PCR may be performed as described below using gene-specific probes (TaqMan Gene Expression Assays Mixture (ASSAYS-ON-DEMAND); Applied Biosystems) and ABI Prism 7900 Sequence Detection System (Perkin-Elmer Applied Biosystems).

Operation may be performed in two-stages, i.e., reverse transcription reaction and PCR reaction. Reverse transcription reaction (the first stage) is performed by adding dNTP, oligo d(T)₁₆ primer, RNase Inhibitor and Multiscribe Reverse Transcriptase (Perkin-Elmer Applied Biosystems) to the resultant RNA, retaining the mixture at 25° C. for 10 min and then heating at 48° C. for 30 min. The reaction is terminated by heating the reaction solution at 95° C. for 5 min.

The resultant cDNA is subjected to the PCR reaction at the second stage. The PCR reaction is performed in a reaction system comprising, for example, 4 ng of cDNA, 1×SYBR PCR buffer, 3 mM MgCl₂, 200 μM each of dATP, dCTP and dGTP, 400 μM dUTP, 200 nM primer pair, 0.01 U/μl AmpErase UNG and 0.025 U/μl AmpliTaq Gold DNA Polymerase (Perkin-Elmer Applied Biosystems). The reaction conditions were as follows: 50° C. for 2 min and 95° C. for 10 min, followed by 40 cycles of 95° C. for 20 sec, 55° C. for 20 sec and 72° C. for 30 sec. Primers and probes may be designed using Primer Expression (Perkin-Elmer Applied Biosystems), for example. Alternatively, TaqMan Gene Expression Assays mixture (ASSAYS-ON-DEMAND; Applied Biosystems) may be used as primers and probes. Comparison of a plurality of samples may be performed by correcting the quantitatively determined values by the mRNA level of a housekeeping gene whose transcription level vary little among samples (preferably, GAPDH, β-actin, 18S ribosomal RNA or the like).

Further, it is preferable to determine in advance the number of blood vessels in a tumor (i.e., the total number of the blood vessels covered with pericytes and the blood vessels not covered with pericytes) using as an indicator the expression of a protein(s) and/or mRNA(s) expressed specifically in vascular endothelial cells. Examples of proteins and/or genes (mRNAs) expressed specifically in vascular endothelial cells include CD31, wVF (von Willebrand Factor), CD34, CD105, CXCR4, CD146, CD133, KDR (VEGF receptor 2) and KIT (Vascular and haematopoietic stem cells: novel targets for anti-angiogenesis therapy? Nature Reviews Cancer, 2, 826-35, 2002). Preferably, CD31 is used. The number of blood vessels may be calculated by such methods as immunochemical methods, in situ hybridization or quantitative RT-PCR, in the same manner as in the determination of the number of blood vessels covered with pericytes.

At this point, it is possible to further improve the accuracy of judgment as to whether a cancer patient is highly sensitive to an angiogenesis inhibitor, by amending the number of those blood vessels covered with pericytes by the total number of blood vessels (i.e., the total number of the blood vessels covered with pericytes and the blood vessels not covered with pericytes). For example, a quotient obtained by dividing the number of those blood vessels covered with pericytes by the total number of blood vessels (i.e., the total number of the blood vessels covered with pericytes and the blood vessels not coated with pericytes) may be used as an indicator.

### 2. Step of Judging Whether or not Cancer Patients are Highly Sensitive to Angiogenesis Inhibitor

In this step, it is possible to judge whether or not cancer patients are highly sensitive to an angiogenesis inhibitor using as an indicator the number of those blood vessels covered with pericytes determined in the previous step. Then, from the result of judgment on the sensitivity, it is possible to predict the antitumor effect of the angiogenesis inhibitor.

In this step, as the number of those blood vessels covered with pericytes, the following values may be used as an indicator, for example: (i) the number of those blood vessels covered with pericytes per unit area in tumor; (ii) the number of those blood vessels covered with pericytes per unit volume in tumor; (iii) the number of those blood vessels covered with pericytes per unit weight in tumor; and (iv) the ratio of the number of those blood vessels covered with pericytes to the total number of blood vessels (i.e., the total number of the blood vessels covered with pericytes and the blood vessels not covered with pericytes) in tumor.

When the number of those blood vessels covered with pericytes in tumor is small, it is possible to judge that the relevant cancer patient is highly sensitive to the angiogenesis inhibitor. On the other hand, when the number of those blood vessels covered with pericytes in the tumor collected from the cancer patient is large, it is possible to judge that the relevant cancer patient is not highly sensitive to the angiogenesis inhibitor.

The expression “when the number of those blood vessels covered with pericytes in tumor is small” means, for example, that the ratio of the number of those blood vessels covered with pericytes to the total number of blood vessels (i.e., the total number of the blood vessels covered with pericytes and the blood vessels not covered with pericytes) is 25% or less, preferably 20% or less, more preferable 15% or less, particularly preferably 10% or less. The expression “when the number of those blood vessels covered with pericytes is large” means, for example, those cases which do not fall under the above-described cases of “when the number of those blood vessels covered with pericytes in tumor is small”.

In the present invention, the major purpose of prediction of antitumor effect is to know how much antitumor effect an angiogenesis inhibitor will produce in cancer patients prior to the administration thereof.

When a cancer patient has been judged highly sensitive to an angiogenesis inhibitor, it is possible to predict that the angiogenesis inhibitor will produce higher antitumor effect in the patient. Cases where higher antitumor effect will be produced include those cases, for example: antitumor effect higher than average antitumor effect in patients with similar symptoms can be expected; antitumor effect higher than the effect in other patients with the same cancer species can be expected; or antitumor effect higher than the effect in patients with other cancer species can be expected.

However, as described later, angiogenesis inhibitor inherently have an angiogenesis inhibitory effect. Therefore, even when a cancer patient has been judged not highly sensitive to an angiogenesis inhibitor, it should not be construed that the relevant angiogenesis inhibitor will produce no antitumor effect.

As another embodiment of the present invention, a method of selecting those patients who are highly sensitive to an angiogenesis inhibitor by using as an indicator the number of those blood vessels covered with pericytes is provided. As described above, when the number of those blood vessels covered with pericytes is small, the relevant patient can be judged highly sensitive to the angiogenesis inhibitor. Therefore, such patients may be selected as patients with high sensitivity to the angiogenesis inhibitor

As still another embodiment of the present invention, the invention provides a method of analyzing the sensitivity to an angiogenesis inhibitor by using as an indicator the number of those blood vessels covered with pericytes and classifying patients according to the results of this analysis. Briefly, in the method of the present invention, it is possible to analyze sensitivity to an angiogenesis inhibitor as described above and classify patients according to the results of this analysis. For example, patients may be classified into a group with a large number of blood vessels covered with pericytes and a group with a small number of blood vessels covered with pericytes.

As still another embodiment of the present invention, the invention provides a method of selecting patients to be administered with an angiogenesis inhibitor by using as an indicator the number of those blood vessels covered with pericytes. Patients who have a small number of blood vessels covered with pericytes are expected to show high sensitivity to the angiogenesis inhibitor. Therefore, these patients are selected as patients to be administered with the angiogenesis inhibitort.

As still another embodiment of the present invention, a method of predicting the therapeutic effect of an angiogenesis inhibitor in a patient by using as an indicator the number of those blood vessels covered with pericytes is provided. In the method of the present invention, when the number of those blood vessels covered with pericytes is small, it can be judged that the relevant patient will show high sensitivity to the angiogenesis inhibitor. Therefore, it is possible to predict that the therapeutic effect of the angiogenesis inhibitor will be high in the patient.

Further, the present invention include a method of evaluating the number of those blood vessels covered with pericytes in a patient, in order to predict the degree of sensitivity of the patient to an angiogenesis inhibitor. The method of evaluation is as described in sub-section 1. above.

In the present step, examples of angiogenesis inhibitors are as described later. Preferably, the angiogenesis inhibitor is 4-(3-chloro-4-(cyclopropylaminocarbonyl)-aminophenoxy)-7-methoxy-6-quinolinecarboxamide, a pharmacologically acceptable salt thereof, or a solvate of the compound or the salt.

The method according to the present invention may be used in order to predict the degree of efficacy of an angiogenesis inhibitor in a patient prior to the administration of the agent to the patient. Also, the method of the present invention makes it possible to select those patients in whom higher effect of the angiogenesis inhibitor can be expected and treat them. Thus, the present invention is clinically very useful.

### 3. Angiogenesis Inhibitors

In the present invention, angiogenesis inhibitors are not particularly limited. Any substance may be used as long as it has inhibitory activity against angiogenesis.

Examples of angiogenesis inhibitors include:

VEGF inhibitors (e.g., VEGF receptor kinase inhibitor, anti-VEGF receptor antibody, anti-VEGF antibody (Cancer Research, 55, 5296-5301, 1995));

FGF (fibroblast growth factor) inhibitors (e.g., FGF receptor kinase inhibitor, anti-FGF receptor antibody, anti-FGF antibody (Cancer Research, 51, 6180-4, 1991));

PDGF (platelet-derived growth factor) inhibitors (e.g., PDGF receptor kinase inhibitor (J. Clinical Investigation, 111, 1287-95), anti-PDGF receptor antibody, anti-PDGF antibody);

EGF (epidermal growth factor) inhibitors (e.g., EGF receptor kinase inhibitor (Cancer Research, 51, 6180-4, 1991), anti-EGF receptor antibody, anti-EGF antibody);

Integrin inhibitors (e.g., αvβ3 integrin inhibitor, αvβ5 integrin inhibitor (Clinical Cancer Research, 6, 3056-61, 2000));

Endogenous inhibitors (e.g., IL-12, trombospondin-1, endostatin, angiostatin (International J. Cancer., 78, 361-5, 1998), COX-2 inhibitor (Annuals of N.Y. Acad. Science., 84-6, 1999));

Matrix metalloprotein inhibitors (International J. Pancreatol., 21, 1-12, 1997);

Other inhibitors (e.g., farnesyltransferase inhibitor, nitric oxide inhibitor, angiotensin-converting enzyme inhibitor, HMG-CoA reductase inhibitor, vascular target inhibitor, methionine aminopeptidase inhibitor (Science, 282, 1324-1327, 1998)); and so on.

Among all, VEGF inhibitors are preferable. More preferable is VEGF receptor kinase inhibitor, anti-VEGF receptor antibody or anti-VEGF antibody. Particularly preferable is VEGF receptor kinase inhibitor.

(A) Definitions of Groups in Compounds

The term “halogen atom” used in the present specification means fluorine atom, chlorine atom, bromine atom or iodine atom.

Preferable examples of “halogen atom” are fluorine atom and chlorine atom.

The term “C₁₋₆ alkyl group” used in the present specification means a straight-chain or branched-chain alkyl group with 1 to 6 carbon atoms. Specific examples include methyl group, ethyl group, 1-propyl group (n-propyl group), 2-propyl group (i-propyl group), 2-methyl-1-propyl group (i-butyl group), 2-methyl-2-propyl group (t-butyl group), 1-butyl group (n-butyl group), 2-butyl group (s(sec)-butyl group), 1-pentyl group, 2-pentyl group, 3-pentyl group, 2-methyl-1-butyl group, 3-methyl-1-butyl group, 2-methyl-2-butyl group, 3-methyl-2-butyl group, 2,2-dimethyl-1-propyl group, 1-hexyl group, 2-hexyl group, 3-hexyl group, 2-methyl-1-pentyl group, 3-methyl-1-pentyl group, 4-methyl-1-pentyl group, 2-methyl-2-pentyl group, 3-methyl-2-pentyl group, 4-methyl-2-pentyl group, 2-methyl-3-pentyl group, 3-methyl-3-pentyl group, 2,3-dimethyl-1-butyl group, 3,3-dimethyl-1-butyl group, 2,2-dimethyl-1-butyl group, 2-ethyl-1-butyl group, 3,3-dimethyl-2-butyl group, 2,3-dimethyl-2-butyl group, or the like.

As preferable examples of “C₁₋₆ alkyl group”, methyl group, ethyl group, 1-propyl group, 2-propyl group, 2-methyl-1-propyl group, 2-methyl-2-propyl group, 1-butyl group, 2-butyl group, 1-pentyl group, 2-pentyl group, 3-pentyl group, 2-methyl-1-butyl group, 3-methyl-1-butyl group, 2-methyl-2-butyl group, 3-methyl-2-butyl group and 2,2-dimethyl-1-propyl group may be enumerated. As more preferable examples, methyl group, ethyl group, 1-propyl group, 2-propyl group, 2-methyl-1-propyl group, 2-methyl-2-propyl group, 1-butyl group and 2-butyl group may be enumerated. As still more preferable examples, methyl group, ethyl group, 1-propyl group and 2-propyl group may be enumerated. As most preferable example, methyl group and ethyl group may be enumerated.

The term “C₁₋₆ alkylene group” used in the present specification means a divalent group which is derived from the above-defined “C₁₋₆ alkyl group” by removing any one hydrogen atom. Specific examples include methylene group, 1,2-ethylene group, 1,1-ethylene group, 1,3-propylene group, tetramethylene group, pentamethylene group, hexamethylene group, or the like.

The term “C₂₋₆ alkenyl group” used in the present specification means a straight-chain or branched-chain alkenyl group with 2 to 6 carbon atoms, having one double bond. Specific examples include ethenyl group (vinyl group), 1-propenyl group, 2-propenyl group (allyl group), 1-butenyl group, 2-butenyl group, 3-butenyl group, pentenyl group, hexenyl group or the like.

The term “C₂₋₆ alkynyl group” used in the present specification means a straight-chain or branched-chain alkynyl group with 2 to 6 carbon atoms, having one triple bond. Specific examples include ethynyl group, 1-propynyl group, 2-propynyl group, 1-butynyl group, 2-butynyl group, 3-butynyl group, pentynyl group, hexynyl group or the like.

The term “C₃₋₈ cycloalkyl group” used in the present specification means a monocyclic or bicyclic saturated aliphatic hydrocarbon group with 3 to 8 carbon atoms. Specific examples include cyclopropyl group, cyclobutyl group, cyclopentyl group, cyclohexyl group, cycloheptyl group, cyclooctyl group, bicyclo[2.1.0]pentyl group, bicyclo[3.1.0]hexyl group, bicyclo[2.1.1]hexyl group, bicyclo[4.1.0]heptyl group, bicyclo[2.2.1]heptyl group (norbornyl group), bicyclo[3.3.0]octyl group, bicyclo[3.2.1]octyl group, bicyclo[2.2.2]octyl group, or the like.

As preferable examples of “C₃₋₈ cycloalkyl group”, cyclopropyl group, cyclobutyl group and cyclopentyl group may be enumerated. As a more preferable example, cyclopropyl group may be given.

The term “C₆₋₁₀ aryl group” used in the present specification means an aromatic hydrocarbon cyclic group with 6 to 10 carbon atoms. Specific examples include phenyl group, 1-naphthyl group, 2-naphthyl group, indenyl group, azulenyl group, or the like.

As a preferable example of “C₆₋₁₀ aryl group”, phenyl group may be given.

The term “heteroatom” used in the present specification means nitrogen atom, oxygen atom or sulfur atom.

The term “5- to 10-membered heteroaryl group” used in the present specification means an aromatic cyclic group in which the ring is composed of 5 to 10 atoms comprising 1 to 5 heteroatoms. Specific examples include furyl group, thienyl group, pyrrolyl group, imidazolyl group, triazolyl group, tetrazolyl group, thiazolyl group, pyrazolyl group, oxazolyl group, isooxazolyl group, isothiazolyl group, furazanyl group, thiadiazolyl group, oxadiazolyl group, pyridyl group, pyrazinyl group, pyridazinyl group, pyrimidinyl group, triazinyl group, purinyl group, pteridinyl group, quinolyl group, isoquinolyl group, naphthyridinyl group, quinoxalinyl group, cinnolinyl group, quinazolinyl group, phthalazinyl group, imidazopyridyl group, imidazothiazolyl group, imidazooxazolyl group, benzothiazolyl group, benzoxazolyl group, benzimidazolyl group, indolyl group, isoindolyl group, indazolyl group, pyrrolopyridyl group, thienopyridyl group, furopyridyl group, benzothiadiazolyl group, benzoxadiazolyl group, pyridopyrimidinyl group, benzofuryl group, benzothienyl group, thienofuryl group, or the like.

As preferable examples of “5- to 10-membered heteroaryl group”, furyl group, thienyl group, pyrrolyl group, imidazolyl group, thiazolyl group, pyrazolyl group, oxazolyl group, isooxazolyl group, isothiazolyl group, pyridyl group and pyrimidinyl group may be enumerated.

The term “3- to 10-membered non-aromatic heterocyclic group” used in the present specification is defined as follows:

(1) the ring thereof is composed of 3 to 10 atoms;
(2) 1 to 2 heteroatoms are included in those atoms;
(3) the ring may contain 1 to 2 double bonds;
(4) the ring may contain 1 to 3 carbonyl groups, sulfinyl groups or sulfonyl groups;
(5) the term means a monocyclic or bicyclic, non-aromatic cyclic group; and when the atoms constituting its ring contain nitrogen atom(s), the nitrogen atom(s) may have a bond extended therefrom.

Specific examples of “3- to 10-membered non-aromatic heterocyclic group” include aziridinyl group, azetidinyl group, pyrrolidinyl group, piperidinyl group, azepanyl group, azocanyl group, piperadinyl group, diazepanyl group, diazocanyl group, diazabicyclo[2.2.1]heptyl group, morpholinyl group, thiomorpholinyl group, 1,1-dioxo-thiomorpholinyl group, oxiranyl group, oxetanyl group, tetrahydrofuryl group, dioxolanyl group, tetrahydropyranyl group, dioxanyl group, tetrahydrothienyl group, tetrahydrothiopyranyl group, oxazolidinyl group, thiazolidinyl group or the like.

As preferable examples of “3- to 10-membered non-aromatic heterocyclic group”, aziridinyl group, azetidinyl group, pyrrolidinyl group, piperidinyl group, azepanyl group, piperadinyl group, diazepanyl group, morpholinyl group, thiomorpholinyl group, 1,1-dioxo-thiomorpholinyl group, tetrahydrofuryl group and tetrahydropyranyl group may be enumerated.

The term “C₁₋₆ alkoxy group” used in the present specification means the above-defined “C₁₋₆ alkyl group” to which an oxygen atom is attached at one end. Specific examples include methoxy group, ethoxy group, 1-propoxy group (n-propoxy group), 2-propoxy group (i-propoxy group), 2-methyl-1-propoxy group (i-butoxy group), 2-methyl-2-propoxy group (t-butoxy group), 1-butoxy group (n-butoxy group), 2-butoxy group (s-butoxy group), 1-pentyloxy group, 2-pentyloxy group, 3-pentyloxy group, 2-methyl-1-butoxy group, 3-methyl-1-butoxy group, 2-methyl-2-butoxy group, 3-methyl-2-butoxy group, 2,2-dimethyl-1-propoxy group, 1-hexyloxy group, 2-hexyloxy group, 3-hexyloxy group, 2-methyl-1-pentyloxy group, 3-methyl-1-pentyloxy group, 4-methyl-1-pentyloxy group, 2-methyl-2-pentyloxy group, 3-methyl-2-pentyloxy group, 4-methyl-2-pentyloxy group, 2-methyl-3-pentyloxy group, 3-methyl-3-pentyloxy group, 2,3-dimethyl-1-butoxy group, 3,3-dimethyl-1-butoxy group, 2,2-dimethyl-1-butoxy group, 2-ethyl-1-butoxy group, 3,3-dimethyl-2-butoxy group, 2,3-dimethyl-2-butoxy group, or the like.

As preferable examples of “C₁₋₆ alkoxy group”, methoxy group, ethoxy group, 1-propoxy group, 2-propoxy group, 2-methyl-1-propoxy group, 2-methyl-2-propoxy group, 1-butoxy group, 2-butoxy group, 1-pentyloxy group, 2-pentyloxy group, 3-pentyloxy group, 2-methyl-1-butoxy group, 3-methyl-1-butoxy group, 2-methyl-2-butoxy group, 3-methyl-2-butoxy group and 2,2-dimethyl-1-propoxy group may be enumerated. As more preferable examples, methoxy group, ethoxy group, 1-propoxy group, 2-propoxy group, 2-methyl-1-propoxy group, 2-methyl-2-propoxy group, 1-butoxy group and 2-butoxy group, may be enumerated. As still more preferable examples, methoxy group, ethoxy group, 1-propoxy group and 2-propoxy group may be enumerated. As most preferable examples, methoxy group and ethoxy group may be enumerated.

The term “C₁₋₆ alkylthio group” used in the present specification means the above-defined “C₁₋₆ allyl group” to which a sulfur atom is attached to at one end. Specific examples include methylthio group, ethylthio group, 1-propylthio group (n-propylthio group), 2-propylthio group (i-propylthio group), 2-methyl-1-propylthio group (i-butylthio group), 2-methyl-2-propylthio group (t-butylthio group), 1-butylthio group (n-butylthio group), 2-butylthio group (s-butylthio group), 1-pentylthio group, 2-pentylthio group, 3-pentylthio group, 2-methyl-1-butylthio group, 3-methyl-1-butylthio group, 2-methyl-2-butylthio group, 3-methyl-2-butylthio group, 2,2-dimethyl-1-propylthio group, 1-hexylthio group, 2-hexylthio group, 3-hexylthio group, 2-methyl-1-pentylthio group, 3-methyl-1-pentylthio group, 4-methyl-1-pentylthio group, 2-methyl-2-pentylthio group, 3-methyl-2-pentylthio group, 4-methyl-2-pentylthio group, 2-methyl-3-pentylthio group, 3-methyl-3-pentylthio group, 2,3-dimethyl-1-butylthio group, 3,3-dimethyl-1-butylthio group, 2,2-dimethyl-1-butylthio group, 2-ethyl-1-butylthio group, 3,3-dimethyl-2-butylthio group, 2,3-dimethyl-2-butylthio group, or the like.

As preferable examples of “C₁₋₆ alkylthio group”, methylthio group, ethylthio group, 1-propylthio group (n-propylthio group), 2-propylthio group (i-propylthio group), 2-methyl-1-propylthio group (i-butylthio group), 2-methyl-2-propylthio group (t-butylthio group), 1-butylthio group (n-butylthio group) and 2-butylthio group (s-butylthio group) may be enumerated.

The term “C₃₋₈ cycloalkoxy group” used in the present specification means the above-defined “C₃₋₈ cycloalkyl group” to which an oxygen atom is attached at one end. Specific examples include cyclopropoxy group, cyclobutoxy group, cyclopentyloxy group, cyclohexyloxy group, cycloheptyloxy group, cyclooctyloxy group, bicyclo[2.1.0]pentyloxy group, bicyclo[3.1.0]hexyloxy group, bicyclo[2.1.1]hexyloxy group, bicyclo[4.1.0]heptyloxy group, bicyclo[2.2.1]heptyloxy group (norbornyloxy group), bicyclo[3.3.0]octyloxy group, bicyclo[3.2.1]octyloxy group, bicyclo[2.2.2]octyloxy group, or the like.

As preferable examples of “C₃₋₈ cycloalkoxy group”, cyclopropoxy group, cyclobutoxy group and cyclopentyloxy group may be enumerated. As a more preferable example, cyclopropoxy group may be given.

The term “mono-C₁₋₆ alkylamino group” used in the present specification means an amino group in which one hydrogen atom is replaced with the above-defined “C₁₋₆ alkyl group”. Specific examples include methylamino group, ethylamino group, 1-propylamino group (n-propylamino group), 2-propylamino group (i-propylamino group), 2-methyl-1-propylamino group (i-butylamino group), 2-methyl-2-propylamino group (t-butylamino group), 1-butylamino group (n-butylamino group), 2-butylamino group (s-butylamino group), 1-pentylamino group, 2-pentylamino group, 3-pentylamino group, 2-methyl-1-butylamino group, 3-methyl-1-butylamino group, 2-methyl-2-butylamino group, 3-methyl-2-butylamino group, 2,2-dimethyl-1-propylamino group, 1-hexylamino group, 2-hexylamino group, 3-hexylamino group, 2-methyl-1-pentylamino group, 3-methyl-1-pentylamino group, 4-methyl-1-pentylamino group, 2-methyl-2-pentylamino group, 3-methyl-2-pentylamino group, 4-methyl-2-pentylamino group, 2-methyl-3-pentylamino group, 3-methyl-3-pentylamino group, 2,3-dimethyl-1-butylamino group, 3,3-dimethyl-1-butylamino group, 2,2-dimethyl-1-butylamino group, 2-ethyl-1-butylamino group, 3,3-dimethyl-2-butylamino group, 2,3-dimethyl-2-butylamino group, or the like.

The term “di-C₁₋₆ alkylamino group” used in the present specification means an amino group in which two hydrogen atoms are replaced with two of the above-defined “C₁₋₆ alkyl group”, respectively. These two C₁₋₆ alkyl groups may be the same or different. Specific examples include N,N-dimethylamino group, N,N-diethylamino group, N,N-di-n-propylamino group, N,N-di-i-propylamino group, N,N-di-n-butylamino group, N,N-di-i-butylamino group, N,N-di-s-butylamino group, N,N-di-t-butylamino group, N-ethyl-N-methylamino group, N-n-propyl-N-methylamino group, N-i-propyl-N-methylamino group, N-n-butyl-N-methylamino group, N-i-butyl-N-methylamino group, N-s-butyl-N-methylamino group, N-t-butyl-N-methylamino group, or the like.

The term “C₂₋₇ acyl group” used in the present specification means a carbonyl group to which the above-defined “C₁₋₆ alkyl group” is attached. Specific examples include acetyl group, propionyl group, isopropionyl group, butylyl group, isobutylyl group, valeryl group, isovaleryl group, pivaloyl group, or the like.

The term “C₂₋₇ alkoxycarbonyl group” used in the present specification means a carbonyl group to which the above-defined “C₁₋₆ alkoxy group” is attached. Specific examples include methoxycarbonyl group, ethoxycarbonyl group, 1-propyloxycarbonyl group, 2-propyloxycarbonyl group, 2-methyl-2-propoxycarbonyl, or the like.

The expression “may have a substituent(s)” used in the present specification means “may have one or a plurality of substituents in any combination at a position(s) capable of substitution”. Specific examples of substituents include halogen atoms, hydroxyl group, thiol group, nitro group, cyano group, formyl group, carboxyl group, amino group, silyl group, methanesulfonyl group, C₁₋₆ alkyl group, C₂₋₆ alkenyl group, C₂₋₆ alkynyl group, C₃₋₈ cycloalkyl group, C₆₋₁₀ aryl group, 5- to 10-membered heteroaryl group, 3- to 10-membered non-aromatic heterocyclic group, C₁₋₆ alkoxy group, C₁₋₆ alkylthio group, C₃₋₈ cycloalkoxy group, mono-C₁₋₆ alkylamino group, di-C₁₋₆ alkylamino group, C₂₋₇ acyl group, C₂₋₇ alkoxycarbonyl group or the like (provided that C₁₋₆ alkyl group, C₂₋₆ alkenyl group, C₂₋₆ alkynyl group, C₃₋₈ cycloalkyl group, C₆₋₁₀ aryl group, 5- to 10-membered heteroaryl group, 3- to 10-membered non-aromatic heterocyclic group, C₁₋₆ alkoxy group, C₁₋₆ alkylthio group, C₃₋₈ cycloalkoxy group, mono-C₁₋₆ alkylamino group, di-C₁₋₆ alkylamino group, C₂₋₇ acyl group and C₂₋₇ alkoxycarbonyl group independently of each other may have 1 to 3 groups selected from the group of substituents described below).

### <Group of Substitutents>

Halogen atom, hydroxyl group, thiol group, nitro group, cyano group, C₁₋₆ alkyl group, C₃₋₈ cycloalkoxy group, C₂₋₆ alkenyl group, C₂₋₆ alkynyl group, C₆₋₁₀ aryl group, 5- to 10-membered heteroaryl group, 3- to 10-membered non-aromatic heterocyclic group, C₁₋₆ alkoxy group and C₁₋₆ alkylthio group.

(B) VEGF Receptor Kinase Inhibitors

In the present invention, the VEGF receptor kinase inhibitor may be, for example, a compound represented by the following general formula (I):

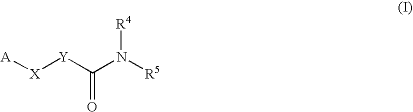

### (i) A

A in general formula (I) is a group represented by one of the following formulas:

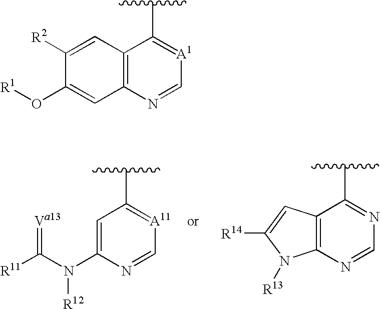

In the above formulas, R¹ is a group represented by a formula —V¹—V²—V³ (where V¹ is a C₁₋₆ alkylene group which may have a substituent(s); V² is a single bond, an oxygen atom, a sulfur atom, a carbonyl group, a sulfinyl group, a sulfonyl group, a group represented by a formula —CONR⁶—, a group represented by a formula —SO₂NR⁶—, a group represented by a formula —NR⁶SO₂—, a group represented by a formula —NR⁶CO— or a group represented by a formula —NR⁶— (where R⁶ is a hydrogen atom, a C₁₋₆ alkyl group which may have a substituent(s) or a C₃₋₈ cycloalkyl group which may have a substituent(s)); and V³ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s); a C₆₋₁₀ aryl group which may have a substituent(s), a 5- to 10-membered heteroaryl group which may have a substituent(s) or a 3- to 10-membered non-aromatic heterocyclic group which may have a substituent(s)).

R² is a cyano group, a C₁₋₆ alkoxy group which may have a substituent(s), a carboxyl group, a C₂₋₇ alkoxycarbonyl group which may have a substituent(s) or a group represented by a formula —CONV^{a11}V^{a12} (where V^{a11} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s); and V^{a12} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s), a hydroxyl group, a C₁₋₆ alkoxy group which may have a substituent(s) or a C₃₋₈ cycloalkoxy group which may have a substituent(s)).

A¹ is a carbon atom or a nitrogen atom which may have a substituent(s).

R¹¹ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s) or a mono-C₁₋₆ alkylamino group which may have a substituent(s).

R¹² is a hydrogen atom or a C₁₋₆ alkyl group which may have a substituent(s).

V^{a13} is an oxygen atom or a sulfur atom.

A¹¹ is a carbon atom or a nitrogen atom which may have a substituent(s).

R¹³ is a hydrogen atom, a C₁₋₆ alkyl group which may have a substituent(s) or a C₃₋₈ cycloalkyl group which may have a substituent(s).

R¹⁴ is a group represented by a formula —V^{a14}—V^{a15} (where V^{a14} is a single bond or a carbonyl group; and V^{a15} is a hydrogen atom, a hydroxyl group, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s), an amino group, a mono-C₁₋₆ allylamino group which may have a substituent(s), a di-C₁₋₆ alkylamino group which may have a substituent(s), a formyl group, a carboxyl group or a C₂₋₇ alkoxycarbonyl group which may have a substituent(s)).

### (ii) X

X in general formula (I) is an oxygen atom or a sulfur atom.

(iii) Y

Y in general formula (I) is a group represented by one of the following formulas:

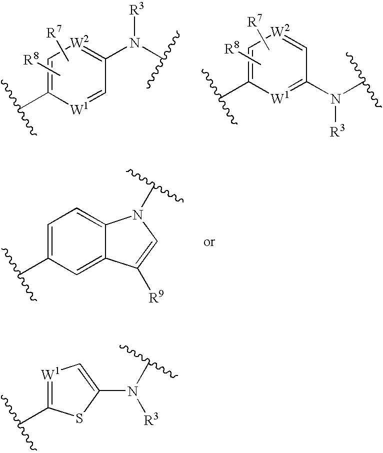

In the above formulas, R³ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₂₋₇ acyl group which may have a substituent(s) or a C₂₋₇ alkoxycarbonyl group which may have a substituent(s).

R⁷ and R⁸ independently of each other represent a hydrogen atom, a halogen atom, a cyano group, a nitro group, an amino group, a C₁₋₆ alkyl group which may have a substituent(s), a C₃₋₈ cycloalkyl group which may have a substituent(s), a C₁₋₆ alkoxy group which may have a substituent(s), a C₁₋₆ alkylthio group which may have a substituent(s), a formyl group, a C₂₋₇ acyl group which may have a substituent(s), a C₂₋₇ alkoxycarbonyl group which may have a substituent(s) or a group represented by a formula —CONV^{d1}V^{d2} (where V^{d1} and V^{d2} independently of each other represent a hydrogen atom or a C₁₋₆ alkyl group which may have a substituent(s)).

R⁹ is a hydrogen atom, a halogen atom or a C₁₋₆ alkyl group which may have a substituent(s).

W¹ and W² independently of each other represent a carbon atom or a nitrogen atom which may have a substituent(s).

### (iv) R⁴

R⁴ in general formula (I) is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₂₋₇ acyl group which may have a substituent(s) or a C₂₋₇ alkoxycarbonyl group which may have a substituent(s).

### (v) R⁵

R⁵ in general formula (I) is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s).

Those compounds represented by general formula (I) may be prepared by known methods. For example, those compounds may be prepared by the method described in any of the following references: WO 02/32872, WO 2004/020434 and WO 2005/063713.

In the present invention, preferably, the VEGF receptor kinase inhibitor is a compound represented by the following general formula (II):

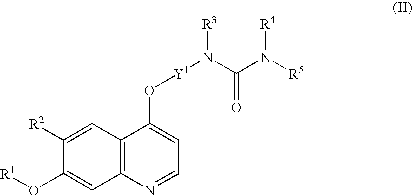

General formula (II) represents preferable examples in the compounds represented by general formula (I).

### (i) R¹

R¹ is as defined above.

As preferable examples of R¹, C₁₋₆ alkyl groups may be given. For example, when V¹ is a C₁₋₆ alkylene group, V² is a single bond; and V³ is a hydrogen atom in the definition of R¹, R¹ is a C₁₋₆ alkyl group. In this case, however, R¹ may have a substituent(s) selected from 3- to 10-membered non-aromatic heterocyclic group which may have C₁₋₆ alkyl group(s), hydroxyl group, C₁₋₆ alkoxy group, amino group, mono-C₁₋₆ alkylamino group and di-C₁₋₆ alkylamino group.

As more preferable examples of R¹, methyl group or a group represented by any of the following formulas may be given:

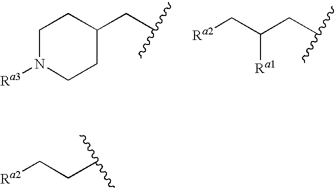

wherein R^{a3} is a methyl group; R^{a1} is a hydrogen atom or a hydroxyl group; and R^{a2} is a methoxy group, an ethoxy group, a 1-pyrrolidinyl group, a 1-piperidinyl group, a 4-morpholinyl group, a dimethylamino group or a diethylamino group.

A still more preferable example of R¹ is methyl group or 2-methoxyethyl group.

### (ii) R²

R² is as defined above.

As preferable examples of R², cyano group or a group represented by a formula CONV^{a11}V^{a12} (where V^{a11} and V^{a12} are as defined above) may be given.

As more preferable examples of R², cyano group or a group represented by a formula —CONHV^{a16} (where V^{a16} is a hydrogen atom, a C₁₋₆ alkyl group, a C₃₋₈ cycloalkyl group, a C₁₋₆ alkoxy group or a C₃₋₈ cycloalkoxy group, provided that V^{a16} may have at least one substituent selected from halogen atoms, cyano group, hydroxyl group and C₁₋₆ alkoxy group) may be given.

As a still more preferable example of R², a group represented by a formula —CONHV^{a17} (where V^{a17} is a hydrogen atom, a C₁₋₆ alkyl group or a C₁₋₆ alkoxy group) may be given.

As a most preferable example of R², a group represented by a formula —CONHV^{a18} (where V^{a18} is a hydrogen atom, a methyl group or a methoxy group) may be given.

(iii) Y¹

Y¹ in general formula (II) is a group represented by one of the following formulas:

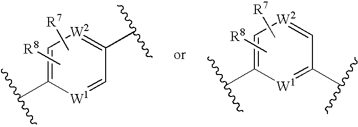

In the above formulas, R⁷, R⁸, W¹ and W² are as defined above.

As a preferable example of Y¹, a group represented by the following formula may be given.

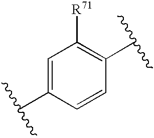

In the above formula, R⁷¹ is a hydrogen atom or a halogen atom.

### (iv) R³ and R⁴

R³ and R⁴ in general formula (II) are as defined above.

As a preferable example of R³ and R⁴, a hydrogen atom may be given for each of them.

### (v) R⁵

R⁵ in general formula (II) is as defined above.

As preferable examples of R⁵, a hydrogen atom, a C₁₋₆ alkyl group, a C₃₋₈ cycloalkyl group or a C₆₋₁₀ aryl group may be given, provided that R⁵ may have a substituent(s) selected from halogen atoms and methanesulfonyl group.

As a more preferable example of R⁵, a methyl group, an ethyl group or a cyclopropyl group may be given.

Preferable examples of the compounds represented by general formula (II) include the following compounds.
N-(4-(6-cyano-7-(2-methoxyethoxy)-4-quinolyl)oxy-2-fluorophenyl)-N′-(4-fluorophenyl)urea,
N-(2-chloro-4-(6-cyano-7-((1-methyl-4-piperidyl)methoxy-4-quinolyl)oxy)phenyl)-N′-cyclopropylurea,
N-(4-((6-cyano-7-(((2R)-3-(diethylamino)-2-hydroxypropyl)oxy)-4-quinolyl)oxy)phenyl)-N′-(4-fluorophenyl)urea,
N-(4-((6-cyano-7-(((2R)-2-hydroxy-3-(1-pyrrolizino)propyl)oxy)-4-quinolyl)oxy)phenyl-N′-(4-fluorophenyl)urea,
4-(3-chloro-4-(cyclopropylaminocarbonyl)aminophenoxy)-7-methoxy-6-quinolinecarboxamide,
4-(3-chloro-4-(cyclopropylaminocarbonyl)aminophenoxy)-7-(2-methoxyethoxy)-6-quinolinecarboxamide,
N6-cyclopropyl-4-(3-chloro-4-(((cyclopropylamino)carbonyl)amino)phenoxy)-7-methoxy-6-quinolinecarboxamide,
N6-(2-methoxyethyl)-4-(3-chloro-4-(((cyclopropylamino)carbonyl)amino)-phenoxy)-7-methoxy-6-quinolinecarboxamide,
N6-(2-fluoroethyl)-4-(3-chloro-4-(((cyclopropylamino)carbonyl)amino)-phenoxy)-7-methoxy-6-quinolinecarboxamide,
N6-methoxy-4-(3-chloro-4-(((cyclopropylamino)carbonyl)amino)phenoxy)-7-methoxy-6-quinolinecarboxamide,
N6-methyl-4-(3-chloro-4-(((cyclopropylamino)carbonyl)amino)phenoxy)-7-methoxy-6-quinolinecarboxamide,
N6-ethyl-4-(3-chloro-4-(((cyclopropylamino)carbonyl)amino)phenoxy)-7-methoxy-6-quinolinecarboxamide,
4-(3-fluoro-4-(cyclopropylaminocarbonyl)aminophenoxy)-7-(2-methoxyethoxy)-6-quinolinecarboxamide,
4-(3-chloro-4-(cyclopropylaminocarbonyl)aminophenoxy)-7-(2-hydroxyethoxy)-6-quinolinecarboxamide,
4-(3-chloro-4-(cyclopropylaminocarbonyl)aminophenoxy)-7-((2S)-2,3-dihydroxypropyl)oxy-6-quinolinecarboxamide,
4-(3-chloro-4-(methylaminocarbonyl)aminophenoxy)-7-methoxy-6-quinolinecarboxamide,
4-(3-chloro-4-(ethylaminocarbonyl)aminophenoxy)-7-methoxy-6-quinolinecarboxamide,
N6-methoxy-4-(3-chloro-4-(((ethylamino)carbonyl)amino)phenoxy)-7-methoxy-6-quinolinecarboxamide,
4-(3-chloro-4-(cyclopropylaminocarbonyl)aminophenoxy)-7-(2-ethoxyethoxy)-6-quinolinecarboxamide,
4-(4-((cyclopropylamino)carbonyl)aminophenoxy)-7-(2-methoxyethoxy)-6-quinolinecarboxamide,
N-(2-fluoro-4-((6-carbamoyl-7-methoxy-4-quinolyl)oxy)phenyl)-N′-cyclopropylurea,
N6-(2-hydroxyethyl)-4-(3-chloro-4-(((cyclopropylamino)carbonyl)amino)-phenoxy)-7-methoxy-6-quinolinecarboxamide,
4-(3-chloro-4-(1-propylaminocarbonyl)aminophenoxy)-7-methoxy-6-quinolinecarboxamide,
4-(3-chloro-4-(cis-2-fluoro-cyclopropylaminocarbonyl)aminophenoxy)-7-methoxy-6-quinolinecarboxamide,
N6-methyl-4-(3-chloro-4-(((cyclopropylamino)carbonyl)amino)phenoxy)-7-(2-methoxyethoxy)-6-quinolinecarboxamide,
N6-methyl-4-(3-chloro-4-(((ethylamino)carbonyl)amino)phenoxy)-7-methoxy-6-quinolinecarboxamide,
4-(3-chloro-4-(cyclopropylaminocarbonyl)aminophenoxy)-7-(2-(4-morpholino)ethoxy)-6-quinolinecarboxamide,
4-(3-chloro-4-(2-fluoroethylaminocarbonyl)aminophenoxy)-7-methoxy-6-quinolinecarboxamide,
N6-((2R)tetrahydro-2-furanylmethyl)-4-(3-chloro-4-(((methylamino)carbonyl)-amino)phenoxy)-7-methoxy-6-quinolinecarboxamide,
4-(3-fluoro-4-(ethylaminocarbonyl)aminophenoxy)-7-methoxy-6-quinolinecarboxamide,
4-(3-chloro-4-(((cyclopropylamino)carbonyl)amino)phenoxy)-7-((2R)-2-hydroxy-3-(1-pyrrolizino)propoxy)-6-quinolinecarboxamide,
N6-methyl-4-(3-chloro-4-(((methylamino)carbonyl)amino)phenoxy)-7-((2R)-3-diethylamino-2-hydroxypropoxy)-6-quinolinecarboxamide,
N6-methyl-4-(3-chloro-4-(((ethylamino)carbonyl)amino)phenoxy)-7-((2R)-3-diethylamino-2-hydroxypropoxy)-6-quinolinecarboxamide,
N6-methyl-4-(3-chloro-4-(((methylamino)carbonyl)amino)phenoxy)-7-((2R)-2-hydroxy-3-(1-pyrrolizino)propoxy)-6-quinolinecarboxamide,
N6-methyl-4-(3-chloro-4-(((ethylamino)carbonyl)amino)phenoxy)-7-((2R)-2-hydroxy-3-(1-pyrrolizino)propoxy)-6-quinolinecarboxamide,
N6-methyl-4-(3-chloro-4-(((methylamino)carbonyl)amino)phenoxy)-7-((1-methyl-4-piperidyl)methoxy)-6-quinolinecarboxamide,
N6-methyl-4-(3-chloro-4-(((ethylamino)carbonyl)amino)phenoxy)-7-((1-methyl-4-piperidyl)methoxy)-6-quinolinecarboxamide,
N-(4-(6-cyano-7-(2-methoxyethoxy)-4-quinolyl)oxy-2-fluorophenyl)-N′-cyclopropylurea,
N-(4-(6-cyano-7-(3-(4-morpholino)propoxy)-4-quinolyl)oxyphenyl)-N′-(3-methylsulfonyl)phenyl)urea,
4-(4-((cyclopropylamino)carbonyl)aminophenoxy)-7-methoxy-6-quinolinecarboxamide,
4-(3-fluoro-4-((2-fluoroethylamino)carbonyl)aminophenoxy)-7-methoxy-6-quinolinecarboxamide,
N6-(2-ethoxyethyl)-4-(3-chloro-4-(((methylamino)carbonyl)amino)phenoxy)-7-methoxy-6-quinolinecarboxamide,
4-(4-(3-ethylureido)-3-fluoro-phenoxy)-7-methoxyquinoline-6-carboxylic acid (2-cyanoethyl)amide, and
N-(4-(6-(2-cyanoethyl)carbamoyl-7-methoxy-4-quinolyl)oxy-2-fluorophenyl)-N′-cyclopropylurea.

Further, a more preferable example of the compound represented by general formula (II) includes:
4-(3-chloro-4-(cyclopropylaminocarbonyl)aminophenoxy-7-methoxy-6-quinolinecarboxamide,
4-(3-chloro-4-(ethylaminocarbonyl)aminophenoxy-7-methoxy-6-quinolinecarboxamide,
N6-methoxy-4-(3-chloro-4-(((cyclopropylamino)carbonyl)amino)phenoxy)-7-methoxy-6-quinolinecarboxamide,
4-(3-chloro-4-(methylaminocarbonyl)aminophenoxy)-7-methoxy-6-quinolinecarboxamide, and
N6-methoxy-4-(3-chloro-4-(((ethylamino)carbonyl)amino)phenoxy)-7-methoxy-6-quinolinecarboxamide.

Further, as a more preferable example of the compound represented by general formula (II), 4-(3-chloro-4-(cyclopropylaminocarbonyl)aminophenoxy-7-methoxy-6-quinolinecarboxamide (see formula (IV)) may be given. As one of the most preferable examples of VEGF receptor kinase inhibitors, the methanesulfonic acid salt of 4-(3-chloro-4-(cyclopropylaminocarbonyl)aminophenoxy-7-methoxy-6-quinolinecarboxamide may be given.

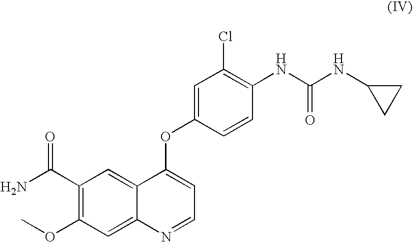

Those compounds represented by general formula (II) may be prepared by known methods. For example, those compounds may be prepared by the method described in WO 02/32872 or WO 2005/063713.

In the present invention, preferably, the VEGF receptor kinase inhibitor is a compound represented by the following general formula (III):

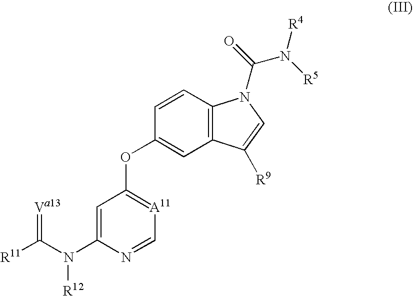

General formula (III) represents preferable examples in the compounds represented by general formula (I).

### (i) R¹¹

R¹¹ is as defined above.

As preferable examples of R¹¹, 3- to 10-membered non-aromatic heterocyclic groups which may have a substituent(s) or mono-C₁₋₆ alkylamino groups which may have a substituent(s) may be given.

As a more preferable example of R¹¹, any one group selected from the groups represented by the following formulas may be given:

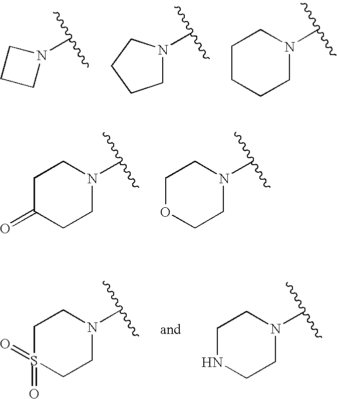

The above group may have a substituent(s) selected from the group of substituents described below.

### [Group of Substituents]

Hydroxyl group, C₁₋₆ alkyl, C₃₋₈ cycloalkyl and groups represented by the formulas:

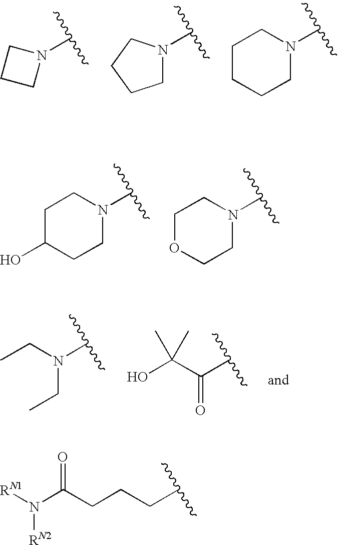

wherein R^{N1} and R^{N2} independently of each other represent a hydrogen atom or a C₁₋₆ alkyl group which may have a substituent(s).

As a still more preferable example of R¹¹, any one group selected from the groups represented by the following formulas may be given:

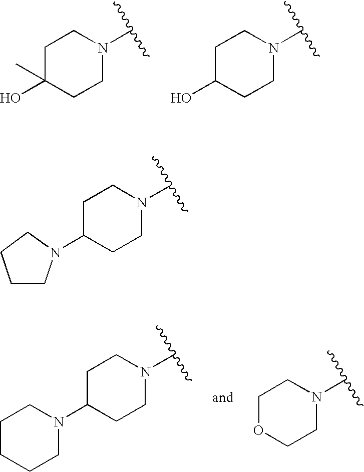

### (ii) R¹²

R¹² is as defined above.

As a preferable example of R¹², a hydrogen atom may be given.

(iii) V^{a13}

V^{a13} is as defined above.

As a preferable example of V^{a13}, an oxygen atom may be given.

### (iv) A¹¹

A¹¹ is as defined above.

As a preferable example of A¹¹, a carbon atom may be given.

### (v) R⁴

R⁴ is as defined above.

As a preferable example of R⁴, a hydrogen atom may be given.

### (vi) R⁵

R⁵ is as defined above.

As a preferable example of R⁵, a C₁₋₆ alkyl group or a C₃₋₈ cycloalkyl group may be given.

As a more preferable of R⁵, a methyl group may be given.

(vii) R⁹

R⁹ is as defined above.

As a preferable example of R⁹, a hydrogen atom may be given.

Preferable examples of the compounds represented by general formula (III) include the following compounds.
5-(2-(((4-hydroxy-4-methylpiperidine-1-yl)carbonyl)amino)pyridine-4-yloxy)-1H-indole-1-carboxylic acid methylamide,
N1-methyl-5-(2-((4-hydroxypiperidino)carbonyl)amino-4-pyridyl)oxy-1H-1-indolecarboxamide,
N1-methyl-5-(2-(((4-pyrrolizine-1-yl)piperidine-1-yl)carbonyl)amino)pyridine-4-yloxy)-1H-1-indolecarboxamide,
N1-methyl-5-(2-(((4-piperidine-1-yl)piperidine-1-yl)carbonyl)amino)pyridine-4-yloxy)-1H-1-indolecarboxamide, and
N4-(4-(1-(methylamino)carbonyl-1H-5-indolyl)oxy-2-pyridyl)-4-morpholinecarboxamide.

The compounds represented by general formula (III) may be prepared by known methods, e.g., the method described in WO 2004/020434.

In the present invention, examples of the VEGF receptor kinase inhibitor include, but are not limited to, the following compounds.

(1) N-(4-bromo-2-fluorophenyl)-6-methoxy-7-[2-(1H-1,2,3-triazole-1-yl)-ethoxy]quinazoline-4-amine (hereinafter, sometimes referred to as “ZD4190”. Cancer Research., 60, 970-975, 2000, Journal of Medicinal Chemistry., 42: 5369-5389, 1999.) (See formula (V) below):

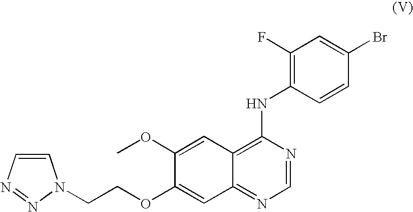

(2) N-(4-bromo-2-fluorophenyl)-6-methoxy-7-[(1-methylpiperidine-4-yl)-methoxy]quinazoline-4-amine (hereinafter, sometimes referred to as “ZD6474” or “vandetanib”. Proc. Am. Assoc. Cancer Research., 42, 583, 2001, Journal of Medicinal Chemistry., 45: 1300-1312, 2002.) (See formula (VI) below):

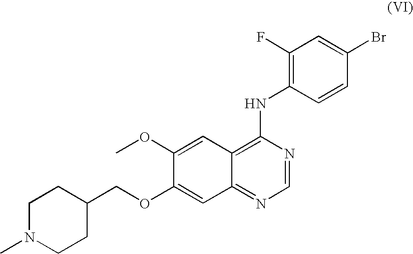

(3) 3-[(2,4-dimethylpyrrol-5-yl)methylene]-2-indolinone (hereinafter, sometimes referred to as “SU5416” or “semaxanib”. Cancer Research., 59, 99-106, 1999, Journal of Medicinal Chemistry., 41: 2588-2603, 1998; U.S. Pat. No. 5,792,783.) (See formula (VII) below):

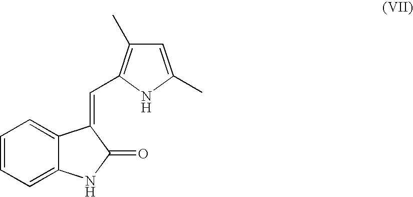

(4) (Z)-3-[(2,4-dimethyl-5-(2-oxo-1,2-dihydroindole-3-ylidenemethyl)-1H-pyrrole-3-yl)-propionic acid (hereinafter, sometimes referred to as “SU6668”. Cancer Research., 60, 4152-4160, 2000, Journal of Medicinal Chemistry., 42: 5120-5130, 1999.) (See formula (VIII) below):

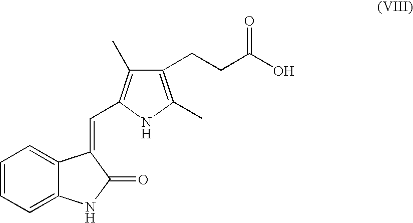

(5) 5-(5-fluoro-2-oxo-1,2-dihydroindole-3-ylidenemethyl)-2,4-dimethyl-1H-pyrrole-3-carboxylic acid (2-diethylaminoethyl)amide (hereinafter, sometimes referred to as “SU11248”. Clinical Cancer Research, 9, 327-337, 2003, Journal of Medicinal Chemistry., 46: 1116-9, 2003; WO 01/060814) (See formula (IX) below):

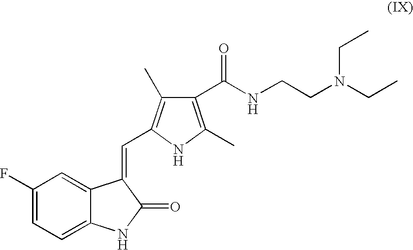

(6) N,N-dimethylglycine-3-{5,6,7,13-tetrahydro-9-[(1-methylethoxy)methyl]-5-oxo-12H-indeno(2,1-a)pyrrolo[3,4-c]carbazole-12-yl}propylester (hereinafter, sometimes referred to as “CEP-7055”. Pro. Am. Assoc. Cancer Research, 43, 1080, 2002, Journal of Medicinal Chemistry., 46: 5375-88, 2003.) (See formula (X) below):

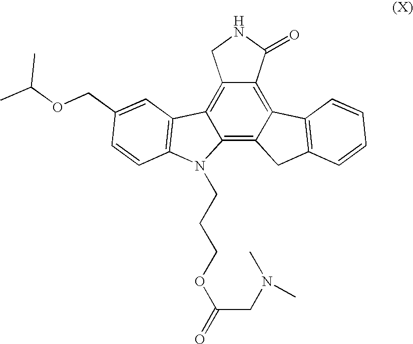

(7) 3-(4-bromo-2,6-difluoro-benzyloxy)-5-[3-(4-pyrrolizine-1-yl-butyl)-ureido]-isothiazole-4-carboxylic acid amide (hereinafter, sometimes referred to as “CP-547,632”. Cancer Research. 63:7301-9, 2003, WO 99/62890.) (See formula (XI) below):

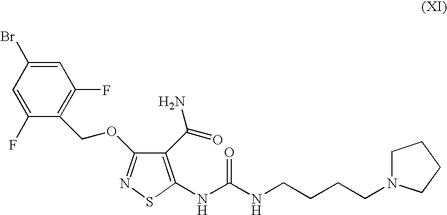

(8) N-{2-chloro-4-[(6,7-dimethoxy-4-quinazolinyl)oxy]phenyl}-N′-propylurea (hereinafter, sometimes referred to as “KRN633”. Molecular Cancer Therapeutics., 3:1639-49, 2004., WO 00/43366.) (See formula (XII) below):

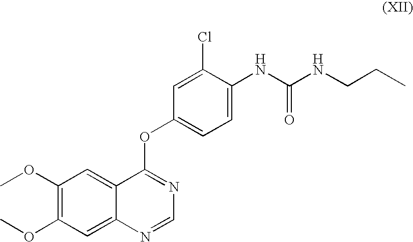

(9) 1-(4-chloroanilino)-4-(4-pyridylmethyl)phthalazine (hereinafter, sometimes referred to as “PTK787/ZK222584” or “vatalanib”. Cancer Research, 60, 2179-2189, 2000, J. Med. Chem., 43:2310-23, 2000; WO 98/35958) (See formula (XIII) below):

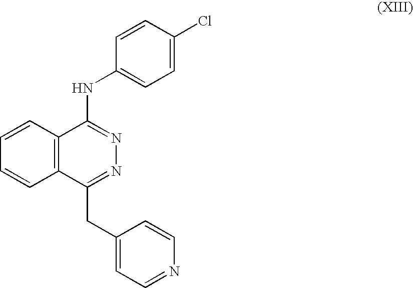

(10) N-{2-chloro-4-[(6,7-dimethoxy-4-quinolyl)oxy]phenyl}-N′-[5-methyl-3-isoxazolyl]urea (hereinafter, sometimes referred to as “KRN951”; WO 2002/088110) (See formula (XIV) below):

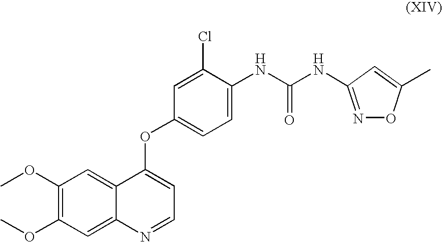

(11) 4-[(4-fluoro-2-methylindole-5-yl)oxy]-6-methoxy-7-[3-(pyrrolizine-1-yl)-propoxy]quinazoline (hereinafter, sometimes referred to as “AZD2171”. Cancer Research. 65:4389-400, 2005; WO 00/47212) (See formula (XV) below):

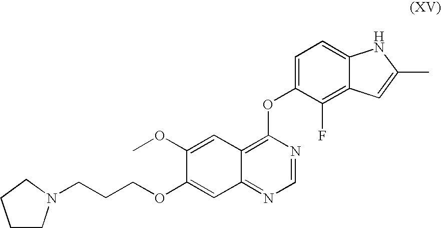

(12) 6-[2-(methylcarbamoyl)phenylsulphanyl]-3-E-[2-(pyridine-2-yl)-ethenyl]indazole (hereinafter, sometimes referred to as “AG013736”. American Journal of Pathology. 165:35-52, 2004; WO 01/002369) (See formula (XVI) below):

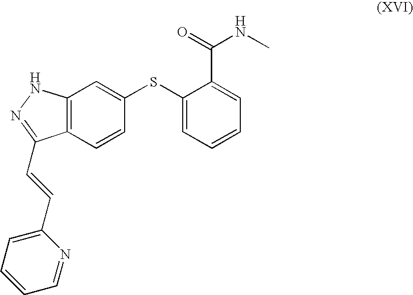

(13) 5-((Z)-(5-fluoro-2-oxo-1,2-dihydro-3H-indole-3-ylidene)methyl)-N-((2S)-2-hydroxy-3-morpholine-4-ylpropyl)-2,4-dimethyl-1H-pyrrole-3-carboxamide (hereinafter, sometimes referred to as “SU14813”. Proceedings of the American Association for Cancer Research, 46, (Abstract 2031), 2005.) (See formula (XVII) below):

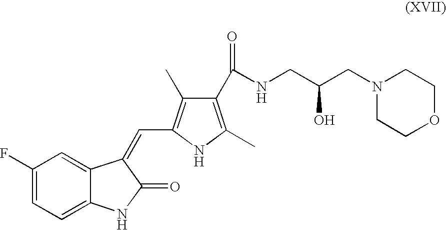

(14) 3-((quinoline-4-ylmethyl)amino)-N-(4-(trifluoromethoxy)phenyl)thiophene-2-carboxamide (hereinafter, sometimes referred to as “OSI930”. Molecular Cancer Therapeutics., 4:1186-1197, 2005.) (See formula (XVIII) below):

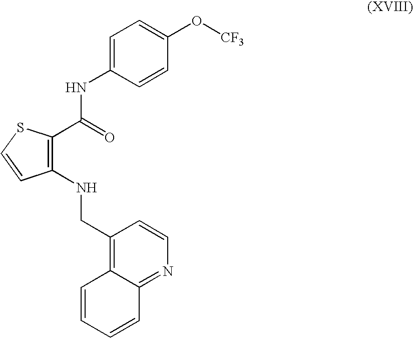

(15) 6-(2,6-dichlorophenyl)-8-methyl-2-phenylamino-8H-pyrido[2,3-d]-pyrimidine-7-one (hereinafter, sometimes referred to as “TKI-28”. Cancer Biol Ther., 4, 2005.) (See formula (XIX) below):

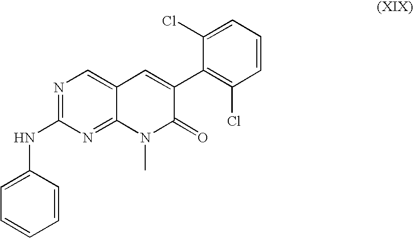

(16) 2-((1,6-dihydro-6-oxo-pyridine-3-ylmethyl)amino)-N-(3-(trifluoromethyl)-phenyl)-3-pyridine-carboxamide (hereinafter, sometimes referred to as “ABP309”. EORTC-NCI-AACR Symp Mol Targets Cancer Ther., 2, (Abstract 172), 2004.) (See formula (XX) below):

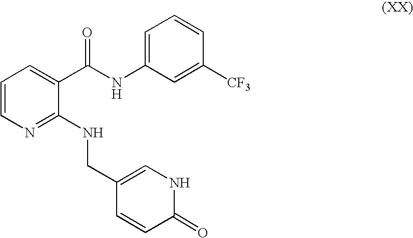

(17) 4-(4-(4-chloro-phenylamino)-furo[2,3-d]pyridazine-7-yloxymethyl)-pyridine-2-carboxylic acid methylamide (hereinafter, sometimes referred to as “BAY 57-9352”. WO 01/23375) (See formula (XXI) below):

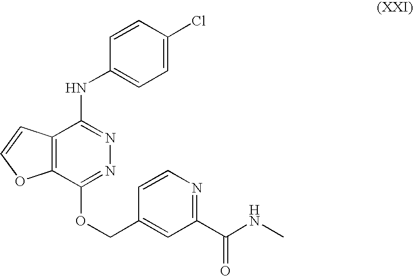

(18) N-(3-trifluoromethyl-4-chlorophenyl)-N′-(4-(2-methylcarbamoylpyridine-4-yl)oxyphenyl)urea (hereinafter, sometimes referred to as “BAY 43-9006” or “sorafenib”. Cancer Research., 64, 7099-7109, 2004, Organic Process Res Dev., 6, 777-81, 2002.) (See formulas (XXII) below):

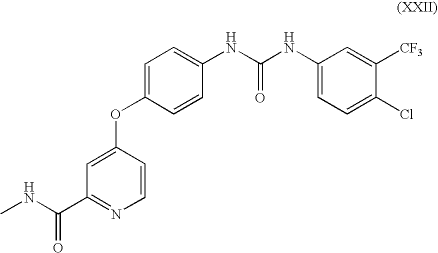

(19) 4-amino-5-fluoro-3-(6-(4-methyl-piperazine-1-yl)-1H-benzimidazole-2-yl)-1H-quinoline-2-one (hereinafter, sometimes referred to as “CHIR258”. Clinical Cancer Research., 11, 3633-3641, 2005.) (See formula (XXIII) below):

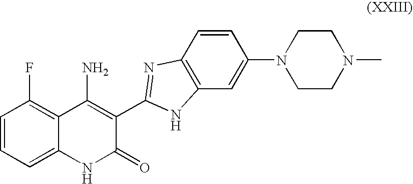

(20) 4-(4-(1-amino-1-methyl-ethyl)-phenyl)-2-(4-(2-morpholine-4-yl-ethyl)-phenylamino)-pyrimidine-5-carbonitrile (hereinafter, sometimes referred to as “JNJ17029259”. Molecular Pharmacology., 66, 635-647, 2004.) (See formula (XXIV) below):

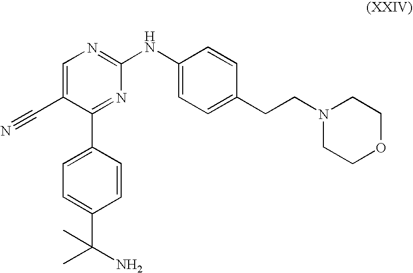

(21) [6-[4-[(4-ethylpiperazine-1-yl)methyl]phenyl]-7H-pyrrolo[2,3-d]pyrimidine-4-yl]-((R)-1-phenylethyl)amine (hereinafter, sometimes referred to as “AEE-788”. Cancer Research., 64, 4931-4941, 2004; Cancer Research., 64, 7977-7984, 2004.) (See formula (XXV) below):

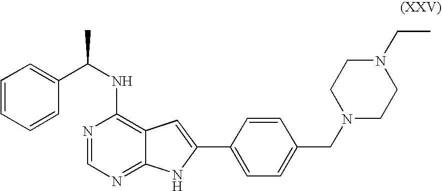

(22) 9-(1-methylethoxy)methyl-12-(3-hydroxypropyl)-6H,7H,13H-indeno[2,1-a]-pyrrole[3,4-c]carbazole-5-one (hereinafter, sometimes referred to as “CEP-5214”. Journal of Medicinal Chemistry., 46, 5375-5388, 2003; Cancer Research., 63, 5978-5991, 2003.) (See formula (XXVI) below):

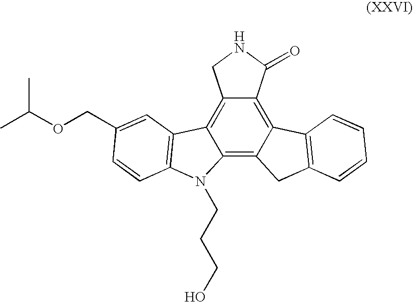

(23) N-(2,4-difluorophenyl)-N′-{4-[(6,7-dimethoxy-4-quinolyl)-oxy]-2-fluorophenyl}urea (hereinafter, sometimes referred to as “KI-8751”. Journal of Medicinal Chemistry., 48, 1359-1366, 2005.) (See formula (XXVII) below):

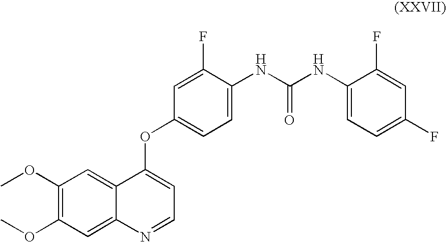

(24) N-[4-(3-amino-1H-indazole-4-yl)phenyl]-N′-(2-fluoro-5-methylphenyl)urea (hereinafter, sometimes referred to as “ABT-869”. Proceedings of the American Association for Cancer Research., 46, 1407, (Abstract 5981), 2005.) (See formula (XXIX) below):

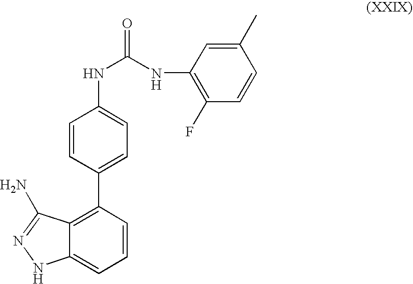

(25) 2-methyl-6-[2-(1-methyl-1H-imidazole-2-yl)-thieno[3,2-b]pyridine-7-yloxy]-benzo[b]thiophene-3-carboxylic acid methylamide (hereinafter, sometimes referred to as “AG-028262”. WO 03/06462; US 2004/009965) (See formula (XXX) below):

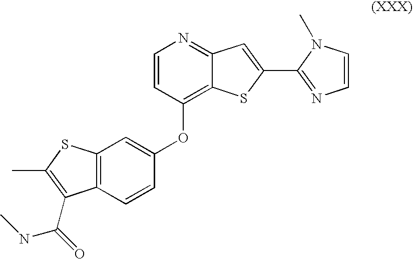

(26) (R)-1-(4-(4-fluoro-2-methyl-1H-indole-5-yloxy)-5-methylpyrrolo[1,2-f]-[1,2,4]triazine-6-yloxy)propane-2-ol (hereinafter, sometimes referred to as “BMS-540215”. Proceedings of the American Association for Cancer Research., 46, (Abstract 3033), 2005.) (See formula (XXXI) below):

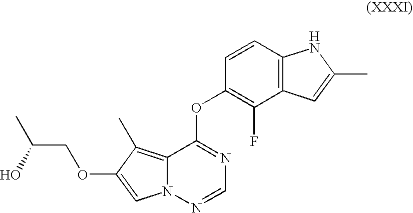

(27) (S)-((R)-1-(4-(4-fluoro-2-methyl-1H-indole-5-yloxy)-5-methylpyrrolo[1,2-f][1,2,4]triazine-6-yloxy)propane-2-ol)2-aminopropanoate (hereinafter, sometimes referred to as “BMS-582664”. Proceedings of the American Association for Cancer Research., 46, (Abstract 3033), 2005.) (See formulas (XXXII) below):

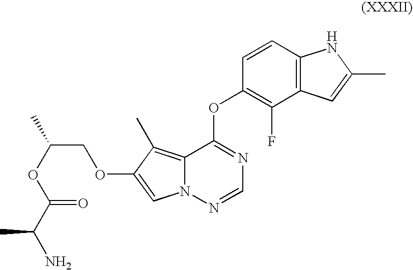

(28) 3-[(4-morpholine-4-yl-phenylamino)-methylene]-1,3-dihydroindole-2-one (hereinafter, sometimes referred to as “AGN-199659”. WO 2003/027102) (See formula (XXXIII) below):

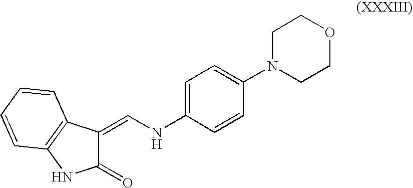

(29) 5-[[4-(2,3-dimethyl-2H-indazole-6-yl)methylamino]pyrimidine-2-yl]amino]-2-methylbenzenesulfonamide (hereinafter, sometimes referred to as “pazopanib” or “GW-786034”. Proc. Am. Soc. Clin. Oncology, (Abstract 3054), 2004.) (See formula (XXXIV) below):

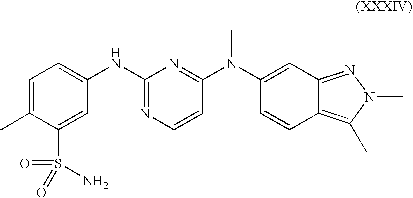

(30) (3Z)-3-[6-(2-morpholine-4-ylethoxy)quinoline-2(1H)-ylidene]-1,3-dihydro-2H-indole-2-one (hereinafter, sometimes referred to as “YM-231146”. Biological and Pharmaceutical Bulletin. 28:2096-2101, 2005.) (See formula (XXXV) below):

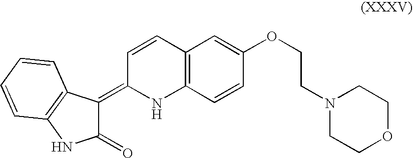

(31) 2-((2-((4-(4-(4-(tert-butyl)anilino)phenoxy)-6-methoxy-7-quinolyl)oxy)ethyl)amino)-1-ethanol (hereinafter, sometimes referred to as “KI-23057”. WO 2003/033472) (See formula (XXXVI) below):

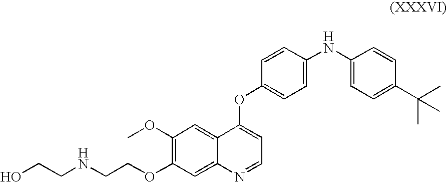

The above-described ZD4190, ZD6474, SU5416, SU6668, SU11248, CEP-7055, CP-547,632, KRN633, PTK787/ZK222584, KRN951, AZD2171, AG013736, SU14813, OSI930, TKI-28, ABP309, BAY 57-9352, BAY 43-9006, CHIR258, JNJ17029259, AEE-788, CEP-5214, KI-8751, ABT-869, AG-028262, BMS-540215, BMS-582664, AGN-199659, pazopanib, YM-231146 and KI-23057 may be prepared by known methods. For example, they may be prepared by the methods described in respective references.

In the present invention, other examples of the VEGF receptor kinase inhibitor include BIBF1120 (WO 01/27081), ZK304709 (Proceedings of the American Association for Cancer Research, 46, (Abstract 5842), 2005), Exe17647 (EORTC-NCI-AACR Symp Mol Targets Cancer Ther., (Abstract 134), 2004), AMG706 (EORTC-NCI-AACR Symp Mol Targets Cancer Ther., 2, (Abstract 151), 2004) and GW-654652 (Blood., 103, 3474-3479, 2004; Proceedings of the American Association for Cancer Research, 44, 9, (Abstract 39), 2003; Proceedings of the American Association for Cancer Research, 44, 9, (Abstract 40), 2003). BIBF1120, ZK304709, Exe17647, AMG706 and GW-654652 may be prepared by known methods.

(C) Anti-VEGF Receptor Antibody

In the present invention, as one example of the VEGF inhibitor, anti-VEGF receptor antibody may be given. Anti-VEGF receptor antibody is an antibody which has affinity for VEGF receptor or a partial fragment thereof. Preferably, this anti-VEGF receptor antibody is a neutralizing antibody that recognizes and binds to VEGF receptor and thereby inhibits the activity of VEGF (such as vascular endothelial cell growth activity). Anti-VEGF receptor antibody may be prepared in the same manner as described later for the preparation of anti-VEGF antibody. Anti-VEGF receptor antibody may be either a polyclonal antibody or a monoclonal antibody. The isotype of the anti-VEGF receptor antibody is not particularly limited. Further, the anti-VEGF receptor antibody may be a fragment of an antibody or a single-chain antibody (see the description of anti-VEGF antibody provided later).

Preferable examples of the anti-VEGF receptor antibody include, but are not limited to, 2C3 antibody (U.S. Pat. No. 6,524,583, U.S. Pat. No. 6,676,941), IMC-1121b (U.S. Pat. No. 6,811,779), IMC-18F1 (Proceedings of the American Association for Cancer Research, 45, 694, (Abstract 3005), 2004), IMC-1C11 (U.S. Pat. No. 5,747,651) and IMC-2C6 (Proceedings of the American Association for Cancer Research, 44, 1479, (Abstract 6454), 2003). 2C3 antibody, IMC-1121b, IMC-18F1, IMC-1C11 and IMC-2C6 may be prepared by known methods. For example, they may be prepared by the methods described in respective references.

(D) Other VEGF Inhibitors

In the present invention, examples of the VEGF inhibitor include PI88, AVE-0005 (Proc. Am. Soc. Clin. Oncology, (Abstract 776), 2003), EG-3306 (Biochem Biophys Res Commun., 302, 793-799, 2003), RPI-4610 (Angiozyme (Registered Trademark), U.S. Pat. No. 5,180,818, U.S. Pat. No. 6,346,398), 2-(8-hydroxy-6-methoxy-1-oxo-1H-2-benzopyran-3-yl)propionic acid (hereinafter, sometimes referred to as “NM-3”; WO 97/48693), 5-[N-methyl-N-(4-octadecyloxyphenyl)acetyl]amino-2-methylthiobenzoic acid (hereinafter, sometimes referred to as “VGA-1155”; Anticancer Research., 24, 3009-3017, 2004) (See formula (LII) below):

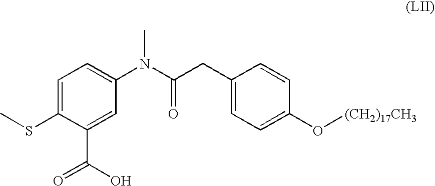

VEGF trap (The Journal of Clinical Endocrinology & Metabolism. 86(7), 3377-3386, 2001) and pegaptanib sodium (Macugen (Registered Trademark)). PI88, AVE-0005, EG-3306, RPI-4610, NM-3, VGA-1155 and VEGF trap may be prepared by known methods. For example, they may be prepared by the methods described in respective references. Pegaptanib sodium may be obtained by purchasing Macugen from Pfizer.

(E) FGF Receptor Kinase Inhibitors

In the present invention, examples of the FGF receptor kinase inhibitor include, but are not limited to, the following compounds.

(1) 1-[2-amino-6-(3,5-dimethoxyphenyl)-pyrido(2,3-d)pyrimidine-7-yl]-3-tert-butylurea (hereinafter, sometimes referred to as “PD166866”; Journal of Medicinal Chemistry., 40, 2296-2303, 1997) (See formula (XXXVII) below):

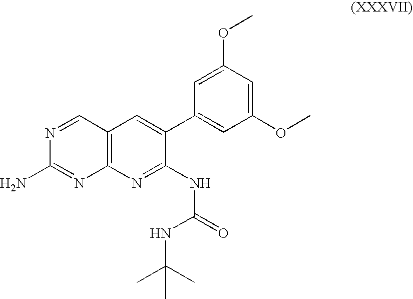

(2) 1-tert-butyl-3-[2-(4-diethylamino)butylamino-6-(3,5-dimethoxyphenyl)-pyrido(2,3-d)pyrimidine-7-yl]urea (hereinafter, sometimes referred to as “PD173074”; EMBO J., 17, 5896-5904, 1998; U.S. Pat. No. 5,733,913) (See formula (XXXVIII) below):

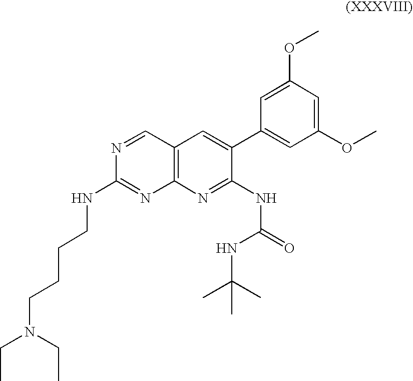

(3) (S)-((R)-1-(4-(4-fluoro-2-methyl-1H-indole-5-yloxy)-5-methylpyrrolo[1,2-f]-[1,2,4]triazine-6-yloxy)propane-2-ol)-2-aminopropanoate (BMS-582664) (See formula (XXXII))

(4) 4-[4-[N-(4-nitrophenyl)carbamoyl]-1-piperazinyl]-6,7-dimethoxyquinazoline (hereinafter, sometimes referred to as “CT-052923”; WO 98/14437) (See formula (XXXIX) below):

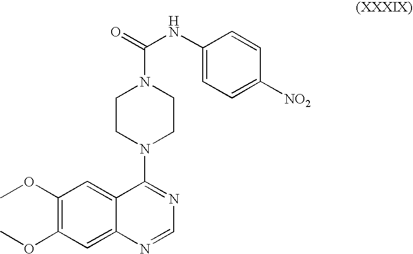

(5) 4-amino-5-fluoro-3-(6-(4-methyl-piperazine-1-yl)-1H-benzimidazole-2-yl)-1H-quinoline-2-one (CHIR258) (See formula (XXIII))

(6) 2-((2-((4-(4-(4-(tert-butyl)anilino)phenoxy)-6-methoxy-7-quinolyl)oxy)ethyl)-amino)-1-ethanol (KI-23057) (See formula (XXXVI))

(7) (Z)-3-[(2,4-dimethyl-5-(2-oxo-1,2-dihydroindole-3-ylidenemethyl)-1H-pyrrole-3-yl)-propionic acid (SU6668) (See formula (VIII))

PD166866, PD173074, BMS-582664, CT-052923, CHIR258, KI-23057 and SU6668 may be prepared by known methods. For example, they may be prepared by the methods described in respective references.

(F) Anti-FGF Receptor Antibody

In the present invention, as one example of the FGF inhibitor, anti-FGF receptor antibody may be given. Anti-FGF receptor antibody is an antibody which has affinity for FGF receptor or a partial fragment thereof. Preferably, this anti-FGF receptor antibody is a neutralizing antibody that recognizes and binds to FGF receptor and thereby inhibits the activity of FGF (such as vascular endothelial cell growth activity). Anti-FGF receptor antibody may be prepared in the same manner as described later for the preparation of anti-VEGF antibody. Anti-FGF receptor antibody may be either a polyclonal antibody or a monoclonal antibody. The isotype of the anti-FGF receptor antibody is not particularly limited. Further, the anti-FGF receptor antibody may be a fragment of an antibody or a single-chain antibody (see the description of anti-VEGF antibody provided later).

(G) PDGF Receptor Kinase Inhibitor

In the present invention, as one example of the PDGF inhibitor, PDGF receptor kinase inhibitor may be given. Examples of the PDGF receptor kinase inhibitor include, but are not limited to, the following compounds.

(1) 4-(4-methylpiperazine-1-ylmethyl)-N-[4-methyl-3-[4-(3-pyridyl)pyrimidine-2-ylamino]phenyl]benzeneamide (hereinafter, sometimes referred to as “imatinib”) (See formula (XL) below):

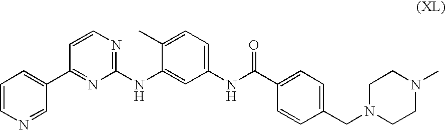

(2) 6-[2-(methylcarbamoyl)phenylsulphanyl]-3-E-[2-(pyridine-2-yl)ethenyl]-indazole (AG013736) (See formula (XVI))

(3) 1-{2-[5-(2-methoxy-ethoxy)-benzoimidazole-1-yl]-quinoline-8-yl}-piperidine-4-ylamine (hereinafter, sometimes referred to as “CP-673451”; WO 2001/040217; Cancer Research., 65, 957-966, 2005.) (See formula (XLI) below):

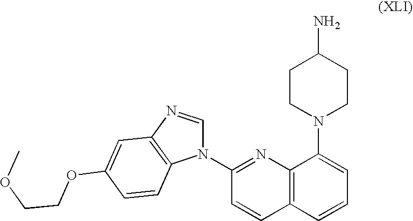

(4) 4-[4-[N-(4-nitrophenyl)carbamoyl]-1-piperazinyl]-6,7-dimethoxyquinazoline (CT-052923) (See formula (XXXIX))

(5) 4-amino-5-fluoro-3-(6-(4-methyl-piperazine-1-yl)-1H-benzimidazole-2-yl)-1H-quinoline-2-one (CHIR258) (See formula (XXIII))

(6) (4-tert-butylphenyl) {4-[(6,7-dimethoxy-4-quinolyl)oxy]phenyl}methaneone (hereinafter, sometimes referred to as “KI-6896”; Bioorganic and Medicinal Chemistry Letters., 7, 2935-2940, 1997.) (See formula (XLIII) below):

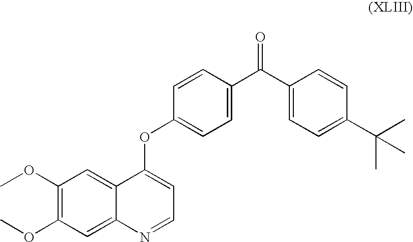

(7) 5-methyl-N-[4-(trifluoromethyl)phenyl]-4-isoxazolecarboxamide (hereinafter, sometimes referred to as “leflunomide”.) (See formula (XLIV) below):

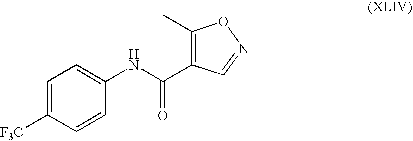

(8) trans-4-[(6,7-dimethoxyquinoxaline-2-yl)amino]cyclohexanol (hereinafter, sometimes referred to as “RPR-127963E”.) (See formula (XLV) below):

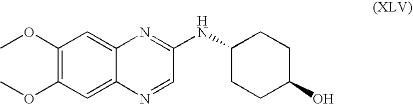

(9) (Z)-3-[(2,4-dimethyl-5-(2-oxo-1,2-dihydroindole-3-ylidenemethyl)-1H-pyrrole-3-yl)-propionic acid (SU6668) (See formula (VIII))

(10) 5-(5-fluoro-2-oxo-1,2-dihydroindole-3-ylidenemethyl)-2,4-dimethyl-1H-pyrrole-3-carboxylic acid (2-diethylaminoethyl)amide (SU11248) (See formula (IX))

(11) 1-(4-chloroanilino)-4-(4-pyridylmethyl)phthalazine (PTK787/ZK222584) (See formula (XIII))

(12) N-[4-(3-amino-1H-indazole-4-yl)phenyl-N′-(2-fluoro-5-methylphenyl)urea (ABT-869) (See formula (XXIX))

Imatinib, AG013736, CP-673451, CT-052923, CHIR258, KI-6896, leflunomide, RPR-127963E, SU6668, SU11248, PTK787/ZK222584 and ABT-869 may be prepared by known methods. For example, they may be prepared by the methods described in respective references.

Imatinib may be obtained by purchasing Glivec (Registered Trademark) from Novartis.

(H) Anti-PDGF Receptor Antibody

In the present invention, as one example of the PDGF inhibitor, anti-PDGF receptor antibody may be given. Anti-PDGF receptor antibody is an antibody which has affinity for PDGF receptor or a partial fragment thereof. Preferably, this anti-PDGF receptor antibody is a neutralizing antibody that recognizes and binds to PDGF receptor and thereby inhibits the activity of PDGF (such as vascular endothelial cell growth activity). Anti-PDGF receptor antibody may be prepared in the same manner as described later for the preparation of anti-VEGF antibody. Anti-PDGF receptor antibody may be either a polyclonal antibody or a monoclonal antibody. The isotype of the anti-PDGF receptor antibody is not particularly limited. Further, the anti-PDGF receptor antibody may be a fragment of an antibody or a single-chain antibody (see the description of anti-VEGF antibody provided later).

(I) EGF Receptor Kinase Inhibitors

In the present invention, as one example of the EGF inhibitor, EGF receptor kinase inhibitor may be given. Specifically, examples of the EGF receptor kinase inhibitor include gefitinib and derivatives thereof. Gefitinib refers to 4-(3-chloro-4-fluorophenylamino)-7-methoxy-6-(3-(4-morpholino)propoxy-quinazoline).

The structural formula thereof is shown in formula (XLVI) below:

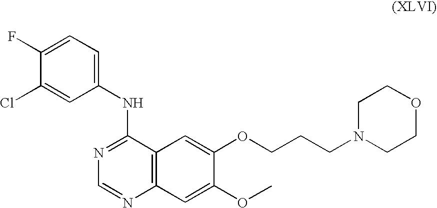

As derivatives of gefitinib, the compounds disclosed in WO 96/33980 may be given.

Gefitinib and derivatives thereof may be prepared by known methods. For example, they may be prepared by the method described in any one of WO 96/33980, Japanese Patent 3040486 and U.S. Pat. No. 5,770,599.

Alternatively, gefitinib may be obtained by purchasing Iressa (Registered Trademark) from Astrazeneca.

In the present invention, further examples of the EGF receptor kinase inhibitor include erlotinib and derivatives thereof. Erlotinib refers to 4-(3-ethynylphenylamino)-6,7-bis(2-methoxyethoxy)-quinazoline. The structural formula thereof is shown in formula (XLVII) below:

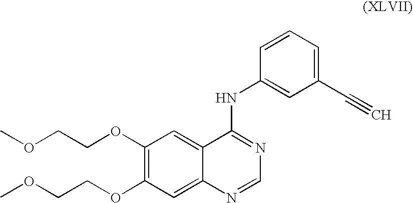

As derivatives of erlotinib, the compounds disclosed in WO 96/30347 may be given.

Erlotinib and derivatives thereof may be prepared by known methods. For example, they may be prepared by the method described in any one of WO 96/30347, Japanese Patent 3088018 and Japanese Patent 3420549.

Alternatively, erlotinib may be obtained by purchasing Tarceva (Registered Trademark) from Genentech.

In the present invention, other examples of the EGF receptor kinase inhibitor include the following compounds.

(1) N-[3-chloro-4-[(3-fluorobenzyl)oxy]phenyl]-6-[5-[[[2-(methylsulfonyl)ethyl]-amino]methyl]furan-2-yl]quinazolin-4-amine (hereinafter, sometimes referred to as “lapatinib”; WO 99/35146; Cancer Research., 64, 6652-6659, 2004) (See formula (XLVIII) below):

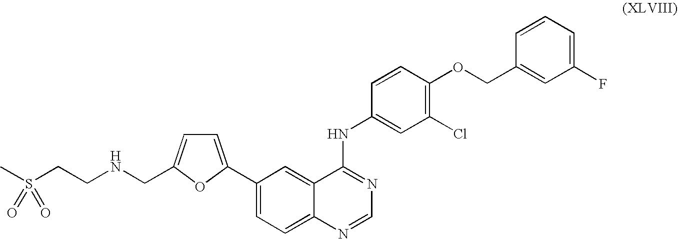

(2) N-[4-[N-(3-chloro-4-fluorophenyl)amino]-7-[3-(4-morpholinyl)propoxy]-quinazolin-6-yl]acrylamide (hereinafter, sometimes referred to as “canertinib”; Clinical Cancer Research., 10:691-700, 2004; WO 2000/31048) (See formula (XLIX) below):

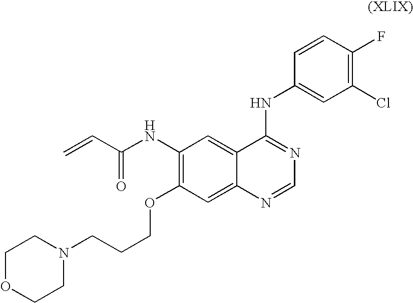

(3) (2E)-N-[4-[(3-chloro-4-fluorophenyl)amino]-3-cyano-7-ethoxy-6-quinolinyl]-4-(dimethylamino)-2-butenamide (hereinafter, sometimes referred to as “pelitinib”; WO 2003/50090) (See formula (L) below):

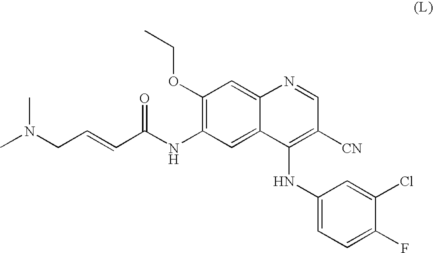

(4) [6-[4-[(4-ethylpiperazine-1-yl)methyl]phenyl]-7H-pyrrolo[2,3-d]pyrimidine-4-yl]-((R)-1-phenylethyl)amine (AEE-788) (See formula (XXV)).

(5) (E)-N-{4-[3-chloro-4-(2-pyridinylmethoxy)anilino]-3-cyano-7-ethoxy-6-quinolinyl}-4-(dimethylamino)-2-butenamide (hereinafter, sometimes referred to as “HKI-272”; Cancer Research., 64, 3958-3965, 2004; Journal of Medicinal Chemistry., 48, 1107-1131, 2005.) (See formula (LI) below):

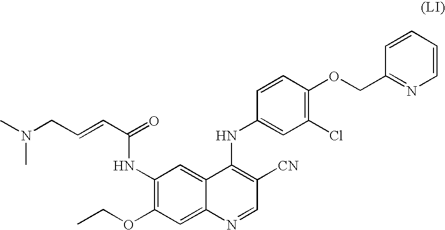

In the present invention, the EGF receptor kinase inhibitor is preferably 4-(3-ethynylphenylamino)-6,7-bis(2-methoxyethoxy)-quinazoline (erlotinib: formula (XLVII) above).

Lapatinib, canertinib, pelitinib, AEE-788 and HKI-272 may be prepared by known methods. For example, they may be prepared the methods described in respective references.

Further, in the present invention, examples of the EGF receptor kinase inhibitor also include ARRY-334543 (Am. Assoc. Cancer Research, A3399, 2005) and MP-412 (Am. Assoc. Cancer Research, A3394, 2005; Am. Assoc. Cancer Research, A3405, 2005). ARRY-334543 and MP-412 may be prepared by known methods.

(J) Anti-EGF Receptor Antibody

In the present invention, as one example of the EGF inhibitor, anti-EGF receptor antibody may be given. Anti-EGF receptor antibody is an antibody which has affinity for EGF receptor or a partial fragment thereof. Preferably, this anti-EGF receptor antibody is a neutralizing antibody that recognizes and binds to EGF receptor and thereby inhibits the activity of EGF (such as vascular endothelial cell growth activity). Anti-EGF receptor antibody may be prepared in the same manner as described later for the preparation of anti-VEGF antibody. Anti-EGF receptor antibody may be either a polyclonal antibody or a monoclonal antibody. The isotype of the anti-EGF receptor antibody is not particularly limited. Further, the anti-EGF receptor antibody may be a fragment of an antibody or a single-chain antibody (see the description of anti-VEGF antibody provided later).

In the present invention, a preferable example of the anti-EGF receptor antibody is cetuximab.

Cetuximab may be prepared by the method described in Japanese Unexamined Patent Publication No. 2002-114710 or No. Hei 2-291295.

Alternatively, cetuximab may be obtained by purchasing Erbitux (Registered Trademark) from Merck.

In the present invention, as another example of the anti-EGF receptor antibody, nimotuzumab may be given. Nimotuzumab may be prepared by the method described in European Patent 203126 or U.S. Pat. No. 5,891,996.

In the present invention, examples of the anti-EGF receptor antibody further include panitumumab (CAS 339177-26-3; Clinical Colorectal Cancer. 2005; 5(1):21-3), matuzumab (CAS 339186-68-4; Curr Opin Mol. Ther. 2004; 6(1):96-103), IMC-11F8 (Am. Assoc. Cancer Research, A5353, 2005) and MDX-447 (ASCO 18: 433, 1999).

(K) Salts and Solvates of Angiogenesis Inhibitors

In the present invention, the angiogenesis inhibitor may form a pharmacologically acceptable salt with acid or base. The above-described angiogenesis inhibitor in the present invention includes such pharmacologically acceptable salts. Examples of salts formed with acid include, but are not limited to, inorganic acid salts such as hydrochlorides, hydrobromates, sulfates and phosphates; and organic acid salts such as formates, acetates, lactates, succinates, fumarates, maleates, citrates, tartrates, stearates, benzoates, methanesulfonates, benzenesulfonates, p-toluenesulfonates and trifluoroacetates. Examples of salts formed with base include, but are not limited to, alkali metal salts such as sodium salts and potassium salts; alkaline earth metal salts such as calcium salts and magnesium salts; organic base salts such as trimethylamine, triethylamine, pyridine, picoline, dicyclohexylamine, N′,N′-dibenzylethylenediamine, arginine and lysine; and ammonium salts.

Further, in the present invention, the angiogenesis inhibitor includes the solvates of these compounds and, when these compounds have optical isomers, the solvates thereof and the optical isomers. Examples of the solvate include, but are not limited to, hydrates and non-hydrates. Hydrates are preferable. Examples of solvents include, but are not limited to, water, alcohols (such as methanol, ethanol, n-propanol) and dimethylformamide.

Further, in the present invention, the angiogenesis inhibitor may be in the form of crystal or non-crystal. When there is crystalline polymorphism, the angiogenesis inhibitor may be a single product of any one of the crystal forms or a mixture of such forms.

In the present invention, the angiogenesis inhibitor also includes those angiogenesis inhibitors which undergo metabolism (such as oxidation, reduction, hydrolysis or conjugation) in the body. Further, in the present invention, the angiogenesis inhibitor also includes those compounds which produce angiogenesis inhibitor in the body as a result of metabolism (such as oxidation, reduction of hydrolysis).

(L) Anti-VEGF Antibody, Anti-FGF Antibody, Anti-PDGF Antibody and Anti-EGF Antibody

In the present invention, anti-VEGF antibody is an antibody which has affinity for VEGF or a partial fragment thereof. Preferably, this anti-VEGF antibody is a neutralizing antibody that recognizes and binds to VEGF and thereby inhibits the vascular endothelial cell growth activity of VEGF. In the present invention, anti-VEGF antibody may be, for example, a polyclonal antibody, monoclonal antibody, chimeric antibody, single-chain antibody (scFV) (Huston et al. (1988) Proc. Natl. Acad. Sci. USA 85: 5879-83; The Pharmacology of Monoclonal Antibody, vol. 113, Rosenburg and Moore ed., Springer Verlag (1994) pp. 269-315), humanized antibody, multispecific antibody (LeDoussal et al. (1992) Int. J. Cancer Suppl. 7: 58-62; Paulus (1985) Behring Inst. Mitt. 78: 118-32; Millstein and Cuello (1983) Nature 305: 537-9; Zimmermann (1986) Rev. Physiol. Biochem. Pharmacol. 105: 176-260; Van Dijk et al. (1989) Int. J. Cancer 43: 944-9), human antibody or an antibody fragment such as Fab, Fab′, F(ab′)₂, Fc or Fv. Preferably, a monoclonal antibody is used. Further, the anti-VEGF antibody may be modified with polyethylene glycol (PEG) or the like, if necessary. Also, the anti-VEGF antibody may be prepared as a fusion protein with β-galactosidase, MBP, GST, GFP or the like. Thus, it is possible to detect the anti-VEGF antibody without using a secondary antibody in methods such as ELISA. Alternatively, the anti-VEGF antibody may be labeled and modified with a substance such as biotin so that the antibody can be recovered with avidin, streptavidin, or the like.

The anti-VEGF antibody may be prepared by conventional methods using VEGF, a partial fragment thereof or a cell expressing one of them as a sensitizing antigen (Current Protocols in Molecular Biology, John Wiley & Sons (1987), Section 11.4-11.13). VEGF or a partial fragment thereof may be a fusion protein with Fc region, GST, MBP, GFP, AP or the like.

Polyclonal antibodies and monoclonal antibodies may be prepared by methods well known to those skilled in the art (Antibodies: A Laboratory Manual, E. Harlow and D. Lane, ed., Cold Spring Harbor Laboratory, Cold Spring Harbor, N.Y., 1988).

Briefly, polyclonal antibodies may be obtained, for example, by administering an antigen to a mammal such as mouse, rabbit, rat, etc., collecting blood from the mammal, isolating antibodies from the collected blood and purifying the antibodies. Methods of immunization are known to those skilled in the art. For example, immunization may be performed by administering an antigen once or more. The antigen (VEGF or a partial fragment thereof) may be dissolved in an appropriate buffer containing a conventionally used adjuvant (such as complete Freund's adjuvant or aluminium hydroxide). However, sometimes, no adjuvant is used depending on the administration routes or other conditions.

One to two months after the final immunization, blood is collected from the mammal and subjected to conventional methods such as centrifugation, precipitation with ammonium sulfate or polyethylene glycol, various chromatographies or the like for separation and purification. As a result, polyclonal antibodies can be obtained as polyclonal antisera.

As a method for producing monoclonal antibodies, the hybridoma method may be given. First, in this method, a mammal is immunized in the same manner as in the production of polyclonal antibodies. After an appropriate number of days from the immunization, it is preferable to collect some blood and to measure the antibody titer by known methods such as ELISA.

Subsequently, the spleen is removed from the immunized animal after sensitization to obtain B cells. The B cells are fused to myeloma cells according to conventional procedures to thereby prepare antibody-producing hybridomas. The myeloma cell used for this purpose is not particularly limited, and known myeloma cells may be used. As a cell fusion method, any of known methods in the art (such as the Sendai virus method, polyethylene glycol method or protoplast method) may be used. The resultant hybridomas may be cultured in HAT medium (medium containing hypoxanthine, aminopterin and thymidine) for an appropriate period according to conventional methods to thereby select appropriate hybridomas. Subsequently, screening for hybridomas producing the antibody of interest is performed. Then, the resultant hybridoma can be cloned.

As a screening method, a known method for antibody detection (such as ELISA or radioimmunoassay) may be used. As a cloning method, a method known in the art (such as the limiting dilution method or FACS method) may be used. The resultant hybridoma may be cultured in an appropriate culture broth or administered to, for example, mouse which is compatible with the hybridoma intraperitoneally. From the thus obtained culture broth or abdominal dropsy, the monoclonal antibody of interest may be isolated and purified by such methods as salting out, ion exchange chromatography, gel filtration, affinity chromatography or the like.

In the present invention, as a preferable example of the anti-VEGF antibody, bevacizumab may be given. Bevacizumab is a human anti-VEGF monoclonal antibody and is sold by Genentech as Avastin (Registered Trademark).

Bevacizumab may be obtained by purchasing Avastin from Genentech.

In the present invention, anti-FGF antibody is an antibody which has affinity for FGF or a partial fragment thereof. Preferably, the anti-FGF antibody is a neutralizing antibody which recognizes and binds to FGF and thereby inhibits the vascular endothelial cell growth activity of FGF. The anti-FGF antibody may be prepared in the same manner as described above for the preparation of anti-VEGF antibody.

In the present invention, anti-PDGF antibody is an antibody which has affinity for PDGF or a partial fragment thereof. Preferably, the anti-PDGF antibody is a neutralizing antibody which recognizes and binds to PDGF and thereby inhibits the vascular endothelial cell growth activity of PDGF. The anti-PDGF antibody may be prepared in the same manner as described above for the preparation of anti-VEGF antibody.

In the present invention, anti-EGF antibody is an antibody which has affinity for EGF or a partial fragment thereof. Preferably, the anti-EGF antibody is a neutralizing antibody which recognizes and binds to EGF and thereby inhibits the vascular endothelial cell growth activity of EGF. The anti-EGF antibody may be prepared in the same manner as described above for the preparation of anti-VEGF antibody.

### 4. Kit

The present invention provides a kit for use in the method of predicting the antitumor effect of an angiogenesis inhibitor, comprising at least one antibody selected from the group consisting of anti-α-SMA antibody, anti-desmin antibody, anti-chondroitin sulfate proteoglycan 4 antibody, anti-calponin antibody, anti-caldesmon antibody and anti-PDGF receptor antibody. Preferably, the antibody is anti-α-SMA antibody. These antibodies may be prepared in the same manner as described above for preparation of anti-VEGF antibody. The antibody contained in the kit may be used in the determination of the number of those blood vessels coated with pericytes in a tumor. The kit of the present invention may also comprise other components conventionally used in common measurement in addition to the above antibody.

Further, the present invention provides a kit for use in the method of predicting the antitumor effect of an angiogenesis inhibitor, comprising a polynucleotide complementary to at least a part of a transcript RNA from at least one gene selected from the group consisting of α-SMA gene, desmin gene, chondroitin sulfate proteoglycan 4 gene, calponin gene, caldesmon gene and PDGF receptor gene. Preferably, the gene is desmin gene. The polynucleotide which is a component of the kit of the present invention is a primer and/or a probe used, for example, in in situ hybridization, Northern blot analysis, DNA microarray, RT-PCR, quantitative RT-PCR or the like. Such a polynucleotide may be designed using, for example, Primer Expression (Perkin-Elmer Applied Biosystems). A desired polynucleotide may be prepared by known methods. The polynucleotide contained in the kit may be used for determining the number of those blood vessels coated with pericytes in a tumor. The kit of the present invention may also comprise other components conventionally used in common measurement, in addition to the above-mentioned polynucleotide.

The base sequences of the above-mentioned genes are registered in various databases. For example, base sequence information may be available with the following GenBank accession numbers.

α-SMA gene: NM_{—}001613

desmin gene: NM_{—}001927

chondroitin sulfate proteoglycan 4 gene: NM_{—}001897

calponin gene: NM_{—}001299

caldesmon gene: NM_{—}033138

PDGF receptor gene: NM_{—}002609

The expression “at least a part of . . . RNA” refers to a base sequence with at least 15 bases, preferably 15-50 bases, more preferably 20-35 bases, still more preferably 20-30 bases. Those skilled in the art could appropriately select the length of the sequence.

Further, the kit of the present invention may comprise an antibody to a protein expressed specifically in vascular endothelial cells and/or a polynucleotide complementary to at least a part of a transcript RNA from a gene expressed specifically in vascular endothelial cells. Such an antibody and a polynucleotide may be used in the determination of the number of blood vessels in a tumor (the total number of the blood vessels coated with pericytes and the blood vessels not coated with pericytes). Examples of proteins and/or genes expressed specifically in vascular endothelial cells include, but are not limited to, CD31, wVF, CD34, CD105, CXCR4, CD146, CD133, KDR (VEGF receptor 2) and KIT.

Hereinbelow, the present invention will be described more specifically with reference to the following Examples. However, the present invention is not limited to these Examples.

### Example 1

### Anti-Tumor Effect of Angiogenesis Inhibitor in Human Cancer Cell Line Subcutaneously Transplanted Models (In Vivo)

Human cancer cell lines A375 (purchased from Dainippon Pharma Co., Ltd.), SEKI, HMV-1 (these two lines were purchased from JCRB cell bank, National Institute of Biomedical Innovation), FEM (granted from Dr. Fodstad, The Norwegian Radiumhospital Research Foundation), LOX (purchased from AntiCancer), AZ-521 (purchased from Japan Health Science Foundation), MDA-MB-468, DLD-1, HCT116, SW620, PC-3, DU145, AsPC-1, H526, MDA-MB-231, SK-Mel-2, Lovo and A431 (these 12 lines were purchased from ATCC) were cultured with RPMI1640 (containing 10% FBS) in a 5% CO₂ incubator until they reached about 80% confluence. After culturing, cells from each line w ere recovered with trypsin-EDTA by conventional procedures. The cells were suspended in phosphate buffer solution to prepare a cell suspension of 1×10⁸ cells/ml or 5×10⁷ cells/ml. Subsequently, 0.1 ml of the cell suspension was subcutaneously transplanted on the lateral side of each nude mouse. After transplantation, when the tumor volume reached about 100-200 mm³, administration of 4-(3-chloro-4-(cyclopropylaminocarbonyl)-aminophenoxy)-7-methoxy-6-quinolinecarboxamide was started (100 mg/kg; twice a day; one week; oral administration). The 4-(3-chloro-4-(cyclopropylaminocarbonyl)-aminophenoxy)-7-methoxy-6-quinolinecarboxamide (a salt of methanesulfonic acid) was prepared based on the disclosure in WO 02/32872 and WO 2005/063713. The major axis and minor axis of tumor were measured with a Degimatic Caliper (Mitsutoyo). Then, tumor volume and ΔT/C were calculated using the following formulas:

Tumor volume(TV)=tumor major axis(mm)×tumor minor axis²(mm²)/2

ΔT/C=(tumor volume at day 8 of administration groups−tumor volume at day 1 of administration groups)/(tumor volume at day 8 of control group−tumor volume at day 1 of control group)×100

In the above formulas, “day 1” means the day when administration started and “day 8” means the 8th day from the start of the administration.

Based on the intensity of the antitumor effect of 4-(3-chloro-4-(cyclopropylaminocarbonyl)aminophenoxy)-7-methoxy-6-quinoline-carboxamide upon these cancer cell lines, they were classified into shrinkage lines, rest lines and proliferation lines. Cancer cell lines which showed ΔT/C<−30% were classified as shrinkage lines; cancer cell lines which showed −30%<ΔT/C<10% were classified as rest lines; and cancer cell lines which showed 10%<ΔT/C were classified as proliferation lines.

### Example 2

### Preparation and Staining of Tumor Tissue Sections from Human Cancer Cell Line Subcutaneously Transplanted Models; and Correlation between the Antitumor Effect of Angiogenesis Inhibitor and the Ratio of the Number of Blood Vessels Covered with Pericytes to the Total Number of Blood Vessels

Human cancer cell lines A375 (purchased from Dainippon Pharma Co., Ltd.), SEKI, HMV-1 (these two strains were purchased from JCRB cell bank, National Institute of Biomedical Innovation), FEM (granted from Dr. Fodstad, The Norwegian Radiumhospital Research Foundation), LOX (purchased from AntiCancer), AZ-521 (purchased from Japan Health Science Foundation), MDA-MB-468, DLD-1, HCT116, SW620, PC-3, DU145, AsPC-1, H526, MDA-MB-231, SK-Mel-2, Lovo and A431 (these 12 strains were purchased from ATCC) were cultured with RPMI1640 (containing 10% FBS) in a 5% CO₂ incubator until they reached about 80% confluence. After culturing, cells from each strain were recovered with trypsin-EDTA by conventional procedures. The cells were suspended in phosphate buffer to prepare a cell suspension of 1×10⁸ cells/ml or 5×10⁷ cells/ml. Subsequently, 0.1 ml of the cell suspension was subcutaneously transplanted on the lateral side of each nude mouse. After transplantation, when the tumor volume reached about 100-200 mm³, the mice were killed with CO₂. Then, the transplanted human tumor was removed by surgical operation. Approximately 5 mm inside of the peripheral part of the tumor tissue was cut with a knife. The thus obtained tumor tissue was embedded in OCT compound, and then frozen with dry ice to prepare a frozen tissue at −80° C. From the resultant tissue, sections 8 μm in thickness were prepared, mounted on slide glass, washed with running water, and then left stationary in cool acetone at 4° for 10 min. Subsequently, these samples were washed 3 times with 0.1% Tween 20-containing 0.01 M phosphate buffer (hereinafter, called “washing PBS”) and reacted in the avidin-blocking solution contained in DAKO Biotin Blockig Kit at room temperature for 10 min. After washing 3 times with the washing PBS, the samples were reacted in the biotin-blocking solution contained in DAKO Biotin Blockig Kit at room temperature for 10 min. After washing in the same manner, the samples were reacted with the normal serum contained in Vector Stain ABC Peroxidase Rat IgG Kit at room temperature for 20 min. After removal of the solution, anti-CD31 antibody (a primary antibody) (designation of the clone: MEC13.3; rat IgG; PharMingen, BD Biosciences) diluted 600-fold with 1% fetal bovine serum-containing 0.1 M phosphate buffer was reacted with the samples at 4° C. overnight. After washing, the biotin-labeled secondary antibody contained in Vector Stain ABC Peroxidase Rat IgG Kit was reacted with the samples at room temperature for 30 min. After washing in the same manner, the avidin reagent (mixture of reagents A and B) contained in Vector Stain ABC Peroxidase Rat IgG Kit was further reacted with the samples at room temperature for 30 min. After washing 3 times with 0.01 M phosphate buffer, the samples were subjected to color formation with DAB to thereby stain CD31.

Subsequently, the samples were washed with running water and then washed 3 times with Tris buffer. Alkaline phosphatase-labeled anti-α-SMA antibody (designation of the clone: 1A4; mouse IgG; SIGMA-ALDRICH) diluted 100-fold with Tris buffer was reacted with the samples at room temperature for 1 hr. After washing 3 times with Tris buffer, the fuchsin solution contained in DAKO LSAB Kit (a solution prepared by mixing each two drops of solutions 3 and 4, agitating for 1 min and adding solution 5 to make a 2 ml solution) was added to the samples for color formation to thereby stain α-SMA.

Under microscopic observation, blood vessels in the thus stained samples were counted. Briefly, the number of all the blood vessels and the number of blood vessels covered with pericytes were counted with a CCD camera Hyper Scope (Keyence) at approximately 5 spots per sample. Average values were obtained from the counting results and were converted into the number of blood vessels per unit area and the number of blood vessels covered with pericytes per unit area. Further, the ratio of blood vessels covered with pericytes to the total number of blood vessels was calculated for each of the cancer cell lines and compared to the antitumor effect of 4-(3-chloro-4-(cyclopropylaminocarbonyl)-aminophenoxy)-7-methoxy-6-quinolinecarboxamide. Statistical analysis was performed by Dunnett's multiple comparison test.

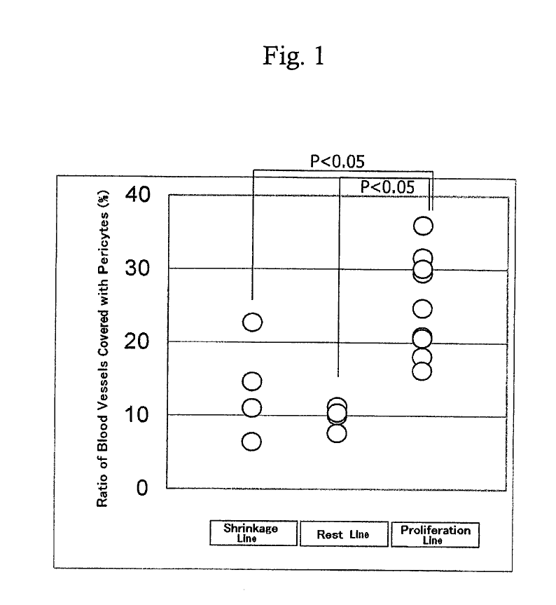

From these results, it has become clear that the antitumor effect of 4-(3-chloro-4-(cyclopropylaminocarbonyl)aminophenoxy)-7-methoxy-6-quinolinecarboxamide correlates with the ratio of blood vessels covered with pericytes in tumor tissues (Table 1 and FIG. 1). Therefore, by determining the ratio of blood vessels covered with pericytes in a tumor and using the resultant number as an indicator, it has become possible to predict the antitumor effect of an angiogenesis inhibitor against the relevant tumor without administering the angiogenesis inhibitor to a patient. Thus, the method of the present invention has made it possible to select those patients who are expected to show greater antitumor effect without actually administering an angiogenesis inhibitor to them, enabling to contribute to patients' QOL.

TABLE 1

| | | | | Ratio of blood vessels |
|---|---|---|---|---|
| Cell Line | Type of Cancer | ΔT/C | Classification | covered with pericytes (%) |
| MDA-MB-468 | Breast cancer | −53% | Shrinkage line | 22.7 |
| MDA-MB-231 | Breast cancer | −33% | Shrinkage line | 6 |
| DU145 | Prostate cancer | −60% | Shrinkage line | 11 |
| AZ-521 | Gastric cancer | −57% | Shrinkage line | 14.6 |
| Lovo | Large bowel cancer | −11% | Rest line | 11 |
| SK-Mel-2 | Melanoma | −2.5% | Rest line | 10 |
| AsPC-1 | Pancreatic cancer | −8% | Rest line | 8 |
| A431 | Epidermoid carcinoma | −8% | Rest line | 10 |
| SW620 | Large bowel cancer | 22% | Proliferation line | 20.6 |
| DLD-1 | Large bowel cancer | 52% | Proliferation line | 29.5 |
| HCT116 | Large bowel cancer | 17% | Proliferation line | 31.6 |
| A375 | Melanoma | 26% | Proliferation line | 20.9 |
| LOX | Melanoma | 42% | Proliferation line | 30 |
| HMV-1 | Melanoma | 19% | Proliferation line | 24.7 |
| SEKI | Melanoma | 110% | Proliferation line | 18.1 |
| FEM | Melanoma | 55% | Proliferation line | 36 |
| PC-3 | Prostate cancer | 54% | Proliferation line | 16 |
| H526 | Small cell lung cancer | 22% | Proliferation line | 81 |

Table 1 shows the antitumor effect of 4-(3-chloro-4-(cyclopropylaminocarbonyl)-aminophenoxy)-7-methoxy-6-quinolinecarboxamide in human cancer cell line transplanted mouse models, the classification of cell lines and the ratio of blood vessels covered with pericytes (%).

### Example 3

### Antitumor Effect of Angiogenesis Inhibitor in Human Cancer Cell Line Subcutaneously Transplanted Models (in vivo)

Human cancer cell lines AsPC-1 and H526 (both strains were purchased from ATCC) were cultured with RPMI1640 (containing 10% FBS) in a 5% CO₂ incubator until they reached about 80% confluence. After culturing, cells from each strain were recovered with trypsin-EDTA by conventional procedures. The cells were suspended in phosphate buffer to prepare a cell suspension of 5×10⁷ cells/ml. Subsequently, 0.1 ml of the cell suspension was subcutaneously transplanted on the lateral side of each nude mouse. After transplantation, when the tumor volume reached about 50-200 mm³, administration of 5-(5-fluoro-2-oxo-1,2-dihydroindole-3-ylidenemethyl)-2,4-dimethyl-1H-pyrrole-3-carboxylic acid (2-diethylaminoethyl)amide (100 mg/kg, once a day, one week, oral administration) was started. The 5-(5-fluoro-2-oxo-1,2-dihydroindole-3-ylidenemethyl)-2,4-dimethyl-1H-pyrrole-3-carboxylic acid (2-diethylaminoethyl)amide was prepared based on the disclosure in WO 01/060814. The major axis and minor axis of tumor were measured with a Degimatic Caliper (Mitsutoyo). Then, tumor volume and relative tumor volume were calculated using the following formulas:

Tumor volume(TV)=tumor major axis(mm)×tumor minor axis²(mm²)/2

Relative tumor volume(RTV)=tumor volume on the measurement day/tumor volume on the starting day of administration

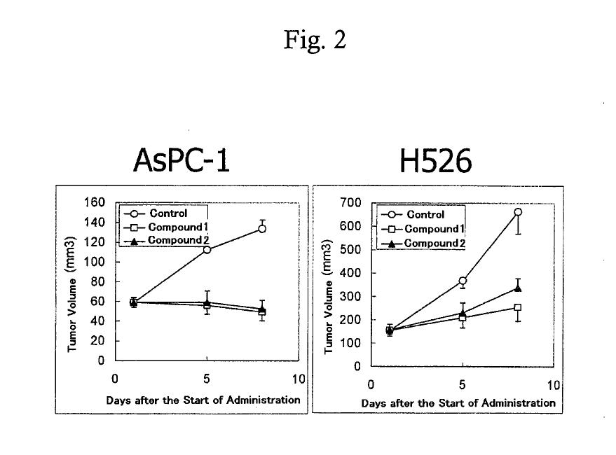

The results revealed that, in the 5-(5-fluoro-2-oxo-1,2-dihydroindole-3-ylidenemethyl)-2,4-dimethyl-1H-pyrrole-3-carboxylic acid (2-diethylaminoethyl)amide (Compound 2) administration group, tumor proliferation rested in AsPC-1 which was classified as a rest cell line in Example 1 and was delayed in H526 which was classified as a proliferation cell line in Example 1, as observed similarly in the 4-(3-chloro-4-(cyclopropylaminocarbonyl)aminophenoxy)-7-methoxy-6-quinoline-carboxamide (Compound 1) administration group (FIG. 2). This means that Compound 2 produces antitumor effect to an extent similar to that extent of the antitumor effect produced by Compound 1. Therefore, it has become clear that not only the antitumor effect of Compound 1 but also the antitumor effect of other angiogenesis inhibitors can be predicted by using as an indicator the ratio of blood vessels covered with pericytes (FIG. 2). This means that it has become possible to predict the antitumor effect of an angiogenesis inhibitor by determining the number of those blood vessels which are covered with pericytes in a tumor and using the resultant number as an indicator, without administering the angiogenesis inhibitor to patient. For this reason, the method of the present invention is capable of selecting those patients who are expected to show greater antitumor effect without administering the angiogenesis inhibitor to these patients and thus contributes to patients' QOL.

### Example 4

### Correlation between the Antitumor Effect of Angiogenesis Inhibitor and the Ratio of the Number of Blood Vessels Covered with Pericytes in Human Cancer Cell Line Subcutaneously Transplanted Models (Quantitative RT-PCR)

1. Purification of Total RNA from Tumor Tissue in Human Cancer Cell Line Subcutaneously Transplanted Models

Human cancer cell lines MDA-MB-468, DLD-1, HCT116, SW620, PC-3, DU145, AsPC-1, H526, MDA-MB-231, MDA-MB-435, SK-OV-3, Lovo, 7860, 22Rv (these 14 strains were purchased from ATCC), HMV-1 (purchased from JCRB cell bank, National Institute of Biomedical Innovation), Colo320DM, A549, A375 (these 3 strains were purchased from Dainippon Pharma Co., Ltd.), FEM (granted from Dr. Fodstad, The Norwegian Radiumhospital Research Foundation) and LOX (purchased from AntiCancer) were cultured with RPMI1640 (containing 10% FBS) in a 5% CO₂ incubator until they reached about 80% confluence. After culturing, cells from each line were recovered with trypsin-EDTA by conventional procedures. The cells were suspended in phosphate buffer to prepare a cell suspension of 1×10⁸ cells/ml or 5×10⁷ cells/ml. Subsequently, 0.1 ml of the cell suspension was subcutaneously transplanted on the lateral side of each nude mouse. After transplantation, when the tumor volume reached about 100-200 mm³, mice were killed with CO₂ and the transplanted human tumor was removed from each mouse by surgical operation. The removed tumor tissue was divided into two portions and individual weights were measured. For one of these two portions, 1 ml of TRIZOL reagent (Invitrogen) was added per 50 mg of the tumor. Then, the tumor was homogenized and stored at −20° C.

Subsequently, 0.2 ml of chloroform (purchased from Junsei Chemical) was added per 1 ml of TRIZOL reagent. The resultant solution was shook and agitated vigorously for 15 sec, left at room temperature for 2-3 min, and then centrifuged (12,000×g, 10 min, 4° C.). After centrifugation, the aqueous layer was transferred to a fresh tube. To this tube, 0.2 ml of isopropyl alcohol (Wako Pure Chemical Industries) was added per 1 ml of the RIZOL reagent used. After leaving at room temperature for 10 min, the tube was centrifuged (12,000×g, 10 min, 4° C.). The resultant precipitate was washed with 75% ethanol (Wako Pure Chemical Industries) and air dried. The thus obtained total RNA was subjected to the subsequent determination.

### 2. Quantitative Determination of RNA

Quantitative RT-PCR was performed as described below using gene-specific probes (TaqMan Gene Expression Assays Mixture (ASSAYS-ON-DEMAND); Applied Biosystems) and ABI Prism 7900 Sequence Detection System (Perkin-Elmer Applied Biosystems).

Operation was performed in two-stages, i.e., reverse transcription reaction and PCR reaction. Reverse transcription reaction (the first stage) was performed by adding to 3 μl of the resultant RNA (100 ng/μl), 6 μl of dNTP, 1.5 μl of oligo d(T)₁₆ primer, 0.6 μl of RNase Inhibitor, 0.75 μl of Multiscribe Reverse Transcriptase, 6.6 μl of 25 mM MgCl₂ (Perkin-Elmer Applied Biosystems) and 6 μl of DEPC water, retaining the mixture at 25° C. for 10 min and then heating at 48° C. for 30 min. The reaction was terminated by heating the reaction solution at 95° C. for 5 min to thereby obtain a cDNA solution for PCR.

The thus obtained cDNA was subjected to the PCR reaction at the second stage. The PCR reaction was performed in a reaction system comprising 5 μl of cDNA solution for PCR diluted 5-fold with DEPC water, 6.25 μl of TaqMan Universal PCR Master Mix, 0.625 μl of 200 nM TaqMan Gene Expression Assays probe and 0.625 μl of H₂O. The reaction conditions were as follows: 50° C. for 2 min and 95° C. for 10 min, followed by 40 cycles of 95° C. for 20 sec, 55° C. for 20 sec and 72° C. for 30 sec. With respect to probes and primers, TaqMan Gene Expression Assays mixture (ASSAYS-ON-DEMAND; Mm00802455_s1; Applied Biosystems) was used for determining desmin, and TaqMan Gene Expression Assays mixture (ASSAYS-ON-DEMAND; Mm00607939_s1; Applied Biosystems) was used for determining β-actin.

### 3. Methods of Data Analysis

For quantitative analysis of individual genes, calibration curves were prepared using mRNA samples of SK-OV-3. Gene expression levels in individual cancer cell lines were determined by calculating Ct (abbreviation of threshold cycle value which means a number of PCR cycles required for a PCR product to reach a specific concentration) from the calibration curve. The expression level of desmin in each cancer cell line amended by the β-actin expression level was taken as the expression level ratio of desmin in each cancer cell line, which was used in comparative analysis. With respect to the classification of individual cell lines, the classification made in Example 1 was used. It should be noted that shrinkage cell lines and rest cell lines were put into one group together and classified as sensitive cell lines.

Comparison between sensitive cell lines and proliferation cell lines was performed by permutation test. When P was <0.05, it was judged as significantly different.

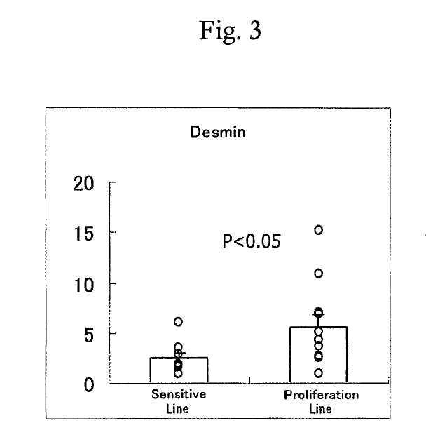

The results revealed that expression of desmin was significantly higher in proliferation cell lines (5.6) than in sensitivity cell lines (2.5) (FIG. 3).

From these results, it has become possible to predict the antitumor effect of an angiogenesis inhibitor by determining the ratio of those blood vessels covered with pericytes in a tumor using as an indicator the expression of a pericyte marker (such as desmin), and then using the number of those blood vessels as an indicator.

### INDUSTRIAL APPLICABILITY

According to the present invention, a method of predicting the antitumor effect of an angiogenesis inhibitor has been provided.

More specifically, it has become possible to predict the antitumor effect of an angiogenesis inhibitor by determining the number of those blood vessels which are covered with pericytes in a tumor and using the determined number as an indicator.

Since the method according to the present invention has made it possible to predict the antitumor effect of an angiogenesis inhibitor without administering the angiogenesis inhibitor to patients, the method allows selection of those patients who are expected to show greater antitumor effect and enables contribution to patients' QOL.

1. A method of predicting the antitumor effect of an angiogenesis inhibitor, comprising the following steps:
a step of determining the ratio of those blood vessels which are covered with pericytes in a tumor; and
a step of judging whether or not a cancer patient is highly sensitive to the angiogenesis inhibitor by using as an indicator the resultant ratio of those blood vessels which are covered with pericytes.

2. The method according to claim 1, which further comprises the following steps:
a step of determining the number of blood vessels in the tumor; and
a step of judging whether or not the cancer patient is highly sensitive to the angiogenesis inhibitor by using as an indicator the ratio of the number of those blood vessels which are covered with pericytes in the tumor to the number of blood vessels in the tumor.

3. A method of predicting the antitumor effect of an angiogenesis inhibitor, comprising the following steps:
a step of determining the number of blood vessels in a tumor and the number of those blood vessels which are covered with pericytes in the tumor; and
a step of judging whether or not a cancer patient is highly sensitive to the angiogenesis inhibitor by using as an indicator the ratio of the number of those blood vessels which are covered with pericytes in the tumor to the number of blood vessels in the tumor.

4. The method according to claim 1, wherein the tumor is a tumor removed from the cancer patient.

5. The method according to claim 1, wherein the determination of the number of those blood vessels which are covered with pericytes is performed by using as an indicator the expression of at least one substance selected from the group consisting of α-SMA, desmin, chondroitin sulfate proteoglycan 4, calponin, caldesmon and PDGF receptor.

6. The method according to claim 1, wherein the determination of the number of those blood vessels which are covered with pericytes is performed by using as an indicator the expression of α-SMA and/or desmin.

7. The method according to claim 1, wherein the determination of the number of those blood vessels which are covered with pericytes is performed by an immunochemical method.

8. The method according to claim 1, wherein the determination of the number of those blood vessels which are covered with pericytes is performed by in situ hybridization.

9. The method according to claim 1, wherein the determination of the number of those blood vessels which are covered with pericytes is performed by quantitative RT-PCR.

10. The method according to claim 2, wherein the determination of the number of blood vessels in the tumor is performed by using as an indicator the expression of at least one substance selected from the group consisting of CD31, wVF, CD34, CD105, CXCR4, CD146, CD133, KDR and KIT.

11. The method according to claim 2, wherein the determination of the number of blood vessels in the tumor is performed by using as an indicator the expression of CD31.

12. The method according to claim 2, wherein the determination of the number of blood vessels in the tumor is performed by an immunochemical method.

13. The method according to claim 2, wherein the determination of the number of blood vessels in the tumor is performed by in istu hybridization.

14. The method according to claim 2, wherein the determination of the number of blood vessels in the tumor is performed by quantitative RT-PCR.

15. The method according to claim 1, wherein the angiogenesis inhibitor is a VEGF receptor kinase inhibitor.

16. The method according to claim 15, wherein the VEGF receptor kinase inhibitor is a compound represented by the following general formula (I), a pharmacologically acceptable salt thereof, or a solvate of said compound or said salt: 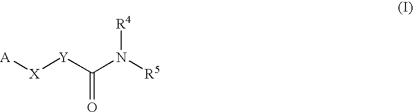
wherein A is a group represented by one of the following formulas: 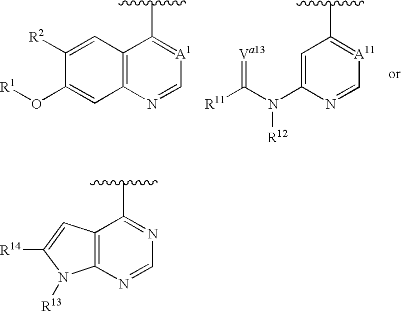
(wherein R¹ is a group represented by a formula —V¹—V²—V³ (where V¹ is a C₁₋₆ alkylene group which may have a substituent(s); V² is a single bond, an oxygen atom, a sulfur atom, a carbonyl group, a sulfinyl group, a sulfonyl group, a group represented by a formula —CONR⁶—, a group represented by a formula —SO₂NR⁶—, a group represented by a formula —NR⁶SO₂—, a group represented by a formula —NR⁶CO— or a group represented by a formula —NR⁶— (where R⁶ is a hydrogen atom, a C₁₋₆ alkyl group which may have a substituent(s) or a C₃₋₈ cycloalkyl group which may have a substituent(s)); and V³ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s); a C₆₋₁₀ aryl group which may have a substituent(s), a 5- to 10-membered heteroaryl group which may have a substituent(s) or a 3- to 10-membered non-aromatic heterocyclic group which may have a substituent(s));
R² is a cyano group, a C₁₋₆ alkoxy group which may have a substituent(s), a carboxyl group, a C₂₋₇ alkoxycarbonyl group which may have a substituent(s) or a group represented by a formula —CONV^{a11}V^{a12} (where V^{a11} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s); a C₆₋₁₀ aryl group which may have a substituent(s), a 5- to 10-membered heteroaryl group which may have a substituent(s) or a 3- to 10-membered non-aromatic heterocyclic group which may have a substituent(s); and V^{a12} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s), a hydroxyl group, a C₁₋₆ alkoxy group which may have a substituent(s) or a C₃₋₈ cycloalkoxy group which may have a substituent(s));
A¹ is a carbon atom or a nitrogen atom which may have a substituent(s);
R¹¹ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s) or a mono-C₁₋₆ alkylamino group which may have a substituent(s);
R¹² is a hydrogen atom or a C₁₋₆ alkyl group which may have a substituent(s);
V^{a13} is an oxygen atom or a sulfur atom;
A¹¹ is a carbon atom or a nitrogen atom which may have a substituent(s);
R¹³ is a hydrogen atom, a C₁₋₆ alkyl group which may have a substituent(s) or a C₃₋₈ cycloalkyl group which may have a substituent(s);
R¹⁴ is a group represented by a formula —V^{a14}—V^{a15} (where V^{a14} is a single bond or a carbonyl group; and V^{a15} is a hydrogen atom, a hydroxyl group, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s), an amino group, a mono-C₁₋₆ alkylamino group which may have a substituent(s), a di-C₁₋₆ alkylamino group which may have a substituent(s), a formyl group, a carboxyl group or a C₂₋₇ alkoxycarbonyl group which may have a substituent(s));
X is an oxygen atom or a sulfur atom;
Y is a group represented by one of the following formulas: 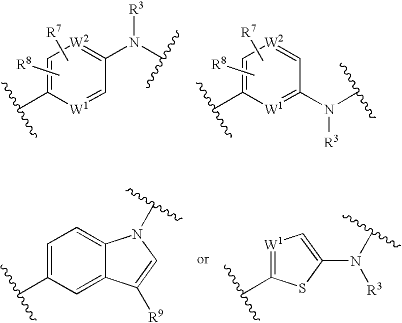
(wherein R³ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₂₋₇ acyl group which may have a substituent(s) or a C₂₋₇ alkoxycarbonyl group which may have a substituent(s);
R⁷ and R⁸ independently of each other represent a hydrogen atom, a halogen atom, a cyano group, a nitro group, an amino group, a C₁₋₆ alkyl group which may have a substituent(s), a C₃₋₈ cycloalkyl group which may have a substituent(s), a C₁₋₆ alkoxy group which may have a substituent(s), a C₁₋₆ alkylthio group which may have a substituent(s), a formyl group, a C₂₋₇ acyl group which may have a substituent(s), a C₂₋₇ alkoxycarbonyl group which may have a substituent(s) or a group represented by a formula —CONV^{d1}V^{d2} (where V^{d1} and V^{d2} independently of each other represent a hydrogen atom or a C₁₋₆ alkyl group which may have a substituent(s));
R⁹ is a hydrogen atom, a halogen atom or a C₁₋₆ alkyl group which may have a substituent(s); and
W¹ and W² independently of each other represent a carbon atom or a nitrogen atom which may have a substituent(s));
R⁴ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₂₋₇ acyl group which may have a substituent(s) or a C₂₋₇ alkoxycarbonyl group which may have a substituent(s); and
R⁵ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s).

17. The method according to claim 15, wherein the VEGF receptor kinase inhibitor is a compound represented by the following general formula (II), a pharmacologically acceptable salt thereof, or a solvate of said compound or said salt: 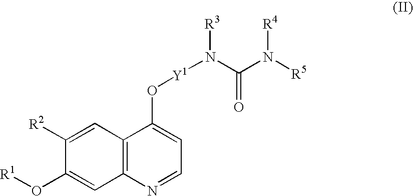
wherein R¹ is a group represented by a formula —V¹—V²—V³ (where V¹ is a C₁₋₆ alkylene group which may have a substituent(s); V² is a single bond, an oxygen atom, a sulfur atom, a carbonyl group, a sulfinyl group, a sulfonyl group, a group represented by a formula —CONR⁶—, a group represented by a formula —SO₂NR⁶—, a group represented by a formula —NR⁶SO₂—, a group represented by a formula —NR⁶CO— or a group represented by a formula —NR⁶— where R⁶ is a hydrogen atom, a C₁₋₆ alkyl group which may have a substituent(s) or a C₃₋₈ cycloalkyl group which may have a substituent(s); and V³ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s); a C₆₋₁₀ aryl group which may have a substituent(s), a 5- to 10-membered heteroaryl group which may have a substituent(s) or a 3- to 10-membered non-aromatic heterocyclic group which may have a substituent(s);
R² is a cyano group, a C₁₋₆ alkoxy group which may have a substituent(s), a carboxyl group, a C₂₋₇ alkoxycarbonyl group which may have a substituent(s) or a group represented by a formula —CONV^{a11}V^{a12} (where V^{a11} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s); a C₆₋₁₀ aryl group which may have a substituent(s), a 5- to 10-membered heteroaryl group which may have a substituent(s) or a 3- to 10-membered non-aromatic heterocyclic group which may have a substituent(s); and V^{a12} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s); a C₆₋₁₀ aryl group which may have a substituent(s), a 5- to 10-membered heteroaryl group which may have a substituent(s), a 3- to 10-membered non-aromatic heterocyclic group which may have a substituent(s), a hydroxyl group, a C₁₋₆ alkoxy group which may have a substituent(s) or a C₃₋₈ cycloalkoxy group which may have a substituent(s));
Y¹ is a group represented by one of the following formulas: 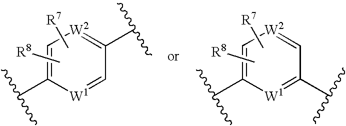
(wherein R⁷ and R⁸ independently of each other represent a hydrogen atom, a halogen atom, a cyano group, a nitro group, an amino group, a C₁₋₆ alkyl group which may have a substituent(s), a C₃₋₈ cycloalkyl group which may have a substituent(s), a C₁₋₆ alkoxy group which may have a substituent(s), a C₁₋₆ alkylthio group which may have a substituent(s), a formyl group, a C₂₋₇ acyl group which may have a substituent(s), a C₂₋₇ alkoxycarbonyl group which may have a substituent(s) or a group represented by a formula —CONV^{d1}V^{d2} (where V^{d1} and V^{d2} independently of each other represent a hydrogen atom or a C₁₋₆ alkyl group which may have a substituent(s)); and
W¹ and W² independently of each other represent a carbon atom or a nitrogen atom which may have a substituent(s));
R³ and R⁴ independently of each other represent a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₂₋₇ acyl group which may have a substituent(s) or a C₂₋₇ alkoxycarbonyl group which may have a substituent(s); and
R⁵ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s).

18. The method according to claim 17, wherein R¹ is a C₁₋₆ alkyl group, provided that R¹ may have at least one substituent selected from the group consisting of 3- to 10-membered non-aromatic heterocyclic group which may have a C₁₋₆ alkyl group(s), hydroxyl group, C₁₋₆ alkoxy group, amino group, mono-C₁₋₆ alkylamino group and di-C₁₋₆ alkylamino group.

19. The method according to claim 17, wherein R¹ is a methyl group or a group represented by any one of the following formulas: 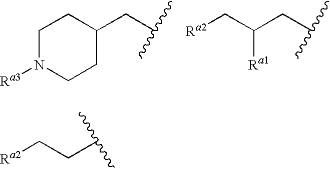
wherein R^{a3} is a methyl group; R^{a1} is a hydrogen atom or a hydroxyl group; and R^{a2} is a methoxy group, an ethoxy group, a 1-pyrrolidinyl group, a 1-piperidinyl group, a 4-morpholinyl group, a dimethylamino group or a diethylamino group.

20. The method according to claim 17, wherein R¹ is a methyl group or a 2-methoxyethyl group.

21. The method according to claim 17, wherein R² is a cyano group or a group represented by a formula —CONV^{a11}V^{a12} (where V^{a11} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s); and V^{a12} is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₁₃ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s), a hydroxyl group, a C₁₋₆ alkoxy group which may have a substituent(s) or a C₃₋₈ cycloalkoxy group which may have a substituent(s)).

22. The method according to claim 17, wherein R² is a cyano group or a group represented by a formula —CONHV^{a16} (where V^{a16} is a hydrogen atom, a C₁₋₆ alkyl group, a C₃₋₈ cycloalkyl group, a C₁₋₆ alkoxy group or a C₃₋₈ cycloalkoxy group, provided that V^{a16} may have at least one substituent selected from the group consisting of halogen atoms, cyano group, hydroxyl group and C₁₋₆ alkoxy group).

23. The method according to claim 17, wherein R² is a group represented by a formula —CONHV^{a17} (where V^{a17} is a hydrogen atom, a C₁₋₆ alkyl group or a C₁₋₆ alkoxy group).

24. The method according to claim 17, wherein R² is a group represented by a formula —CONHV^{a18} (where V^{a18} is a hydrogen atom, a methyl group or a methoxy group).

25. The method according to claim 17, wherein Y¹ is a group represented by the following formula: 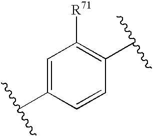
where R⁷¹ is a hydrogen atom or a halogen atom.

26. The method according to claim 17, wherein R³ and R⁴ individually represent a hydrogen atom.

27. The method according to claim 17, wherein R⁵ is a hydrogen atom, a C₁₋₆ alkyl group, a C₃₋₈ cycloalkyl group or a C₈₋₁₀ aryl group, provided that R⁵ may have at least one substituent selected from the group consisting of halogen atoms and methanesulfonyl group.

28. The method according to claim 17, wherein R⁵ is a methyl group, an ethyl group or a cyclopropyl group.

29. The method according to claim 15, wherein the VEGF receptor kinase inhibitor is at least one compound selected from the group consisting of:
N-(4-(6-cyano-7-(2-methoxyethoxy)-4-quinolyl)oxy-2-fluorophenyl)-N′-(4-fluorophenyl)urea,
N-(2-chloro-4-((6-cyano-7-((1-methyl-4-piperidyl)methoxy-4-quinolyl)oxy)phenyl)-N′-cyclopropylurea,
N-(4-((6-cyano-7-(((2R)-3-(diethylamino)-2-hydroxypropyl)oxy)-4-quinolyl)oxy)phenyl)-N′-(4-fluorophenyl)urea,
N-(4-((6-cyano-7-(((2R)-2-hydroxy-3-(1-pyrrolizino)propyl)oxy)-4-quinolyl)oxy)phenyl-N′-(4-fluorophenyl)urea,
4-(3-chloro-4-(cyclopropylaminocarbonyl)aminophenoxy)-7-methoxy-6-quinolinecarboxamide,
4-(3-chloro-4-(cyclopropylaminocarbonyl)aminophenoxy)-7-(2-methoxyethoxy)-6-quinolinecarboxamide,
N6-cyclopropyl-4-(3-chloro-4-(((cyclopropylamino)carbonyl)amino)phenoxy)-7-methoxy-6-quinolinecarboxamide,
N6-(2-methoxyethyl)-4-(3-chloro-4-(((cyclopropylamino)carbonyl)amino)-phenoxy)-7-methoxy-6-quinolinecarboxamide,
N6-(2-fluoroethyl)-4-(3-chloro-4-(((cyclopropylamino)carbonyl)amino)-phenoxy)-7-methoxy-6-quinolinecarboxamide,
N6-methoxy-4-(3-chloro-4-(((cyclopropylamino)carbonyl)amino)phenoxy)-7-methoxy-6-quinolinecarboxamide,
N6-methyl-4-(3-chloro-4-(((cyclopropylamino)carbonyl)amino)phenoxy)-7-methoxy-6-quinolinecarboxamide,
N6-ethyl-4-(3-chloro-4-(((cyclopropylamino)carbonyl)amino)phenoxy)-7-methoxy-6-quinolinecarboxamide,
4-(3-fluoro-4-(cyclopropylaminocarbonyl)aminophenoxy)-7-(2-methoxyethoxy)-6-quinolinecarboxamide,
4-(3-chloro-4-(cyclopropylaminocarbonyl)aminophenoxy)-7-(2-hydroxyethoxy)-6-quinolinecarboxamide,
4-(3-chloro-4-(cyclopropylaminocarbonyl)aminophenoxy)-7-((2S)-2,3-dihydroxypropyl)oxy-6-quinolinecarboxamide,
4-(3-chloro-4-(methylaminocarbonyl)aminophenoxy)-7-methoxy-6-quinolinecarboxamide,
4-(3-chloro-4-(ethylaminocarbonyl)aminophenoxy)-7-methoxy-6-quinolinecarboxamide,
N6-methoxy-4-(3-chloro-4-(((ethylamino)carbonyl)amino)phenoxy)-7-methoxy-6-quinolinecarboxamide,
4-(3-chloro-4-(cyclopropylaminocarbonyl)aminophenoxy)-7-(2-ethoxyethoxy)-6-quinolinecarboxamide,
4-(4-((cyclopropylamino)carbonyl)aminophenoxy)-7-(2-methoxyethoxy)-6-quinolinecarboxamide,
N-(2-fluoro-4-((6-carbamoyl-7-methoxy-4-quinolyl)oxy)phenyl)-N′-cyclopropylurea,
N6-(2-hydroxyethyl)-4-(3-chloro-4-(((cyclopropylamino)carbonyl)amino)-phenoxy)-7-methoxy-6-quinolinecarboxamide,
4-(3-chloro-4-(1-propylaminocarbonyl)aminophenoxy)-7-methoxy-6-quinolinecarboxamide,
4-(3-chloro-4-(cis-2-fluoro-cyclopropylaminocarbonyl)aminophenoxy)-7-methoxy-6-quinolinecarboxamide,
N6-methyl-4-(3-chloro-4-(((cyclopropylamino)carbonyl)amino)phenoxy)-7-(2-methoxyethoxy)-6-quinolinecarboxamide,
N6-methyl-4-(3-chloro-4-(((ethylamino)carbonyl)amino)phenoxy)-7-methoxy-6-quinolinecarboxamide,
4-(3-chloro-4-(cyclopropylaminocarbonyl)aminophenoxy)-7-(2-(4-morpholino)ethoxy)-6-quinolinecarboxamide,
4-(3-chloro-4-(2-fluoroethylaminocarbonyl)aminophenoxy)-7-methoxy-6-quinolinecarboxamide,
N6-((2R)tetrahydro-2-furanylmethyl)-4-(3-chloro-4-(((methylamino)carbonyl)-amino)phenoxy)-7-methoxy-6-quinolinecarboxamide,
4-(3-fluoro-4-(ethylaminocarbonyl)aminophenoxy)-7-methoxy-6-quinolinecarboxamide,
4-(3-chloro-4-(((cyclopropylamino)carbonyl)amino)phenoxy)-7-((2R)-2-hydroxy-3-(1-pyrrolizino)propoxy)-6-quinolinecarboxamide,
N6-methyl-4-(3-chloro-4-(((methylamino)carbonyl)amino)phenoxy)-7-((2R)-3-diethylamino-2-hydroxypropoxy)-6-quinolinecarboxamide,
N6-methyl-4-(3-chloro-4-(((ethylamino)carbonyl)amino)phenoxy)-7-((2R)-3-diethylamino-2-hydroxypropoxy)-6-quinolinecarboxamide,
N6-methyl-4-(3-chloro-4-(((methylamino)carbonyl)amino)phenoxy)-7-((2R)-2-hydroxy-3-(1-pyrrolizino)propoxy)-6-quinolinecarboxamide,
N6-methyl-4-(3-chloro-4-(((ethylamino)carbonyl)amino)phenoxy)-7-((2R)-2-hydroxy-3-(1-pyrrolizino)propoxy)-6-quinolinecarboxamide,
N6-methyl-4-(3-chloro-4-(((methylamino)carbonyl)amino)phenoxy)-7-((1-methyl-4-piperidyl)methoxy)-6-quinolinecarboxamide,
N6-methyl-4-(3-chloro-4-(((ethylamino)carbonyl)amino)phenoxy)-7-((1-methyl-4-piperidyl)methoxy)-6-quinolinecarboxamide,
N-(4-(6-cyano-7-(2-methoxyethoxy)-4-quinolyl)oxy-2-fluorophenyl)-N′-cyclopropylurea,
N-(4-(6-cyano-7-(3-(4-morpholino)propoxy)-4-quinolyl)oxyphenyl)-N′-(3-methylsulfonyl)phenyl)urea,
4-(4-((cyclopropylamino)carbonyl)aminophenoxy)-7-methoxy-6-quinolinecarboxamide,
4-(3-fluoro-4-((2-fluoroethylamino)carbonyl)aminophenoxy)-7-methoxy-6-quinolinecarboxamide,
N6-(2-ethoxyethyl)-4-(3-chloro-4-(((methylamino)carbonyl)amino)phenoxy)-7-methoxy-6-quinolinecarboxamide,
4-(4-(3-ethylureido)-3-fluoro-phenoxy)-7-methoxyquinoline-6-carboxylic acid (2-cyanoethyl)amide, and
N-(4-(6-(2-cyanoethyl)carbamoyl-7-methoxy-4-quinolyl)oxy-2-fluorophenyl)-N′-cyclopropylurea;
or a pharmacologically acceptable salt of said compound, or a solvate of said compound or said salt.

30. The method according to claim 15, wherein the VEGF receptor kinase inhibitor is at least one compound selected from the group consisting of:
4-(3-chloro-4-(cyclopropylaminocarbonyl)aminophenoxy)-7-methoxy-6-quinolinecarboxamide,
4-(3-chloro-4-(ethylaminocarbonyl)aminophenoxy)-7-methoxy-6-quinolinecarboxamide,
N6-methoxy-4-(3-chloro-4-(((cyclopropylamino)carbonyl)amino)phenoxy)-7-methoxy-6-quinolinecarboxamide,
4-(3-chloro-4-(methylaminocarbonyl)aminophenoxy)-7-methoxy-6-quinolinecarboxamide, and
N6-methoxy-4-(3-chloro-4-(((ethylamino)carbonyl)amino)phenoxy)-7-methoxy-6-quinolinecarboxamide,
or a pharmacologically acceptable salt of said compound, or a solvate of said compound or said salt.

31. The method according to claim 15, wherein the VEGF receptor kinase inhibitor is 4-(3-chloro-4-(cyclopropylaminocarbonyl)aminophenoxy)-7-methoxy-6-quinolinecarboxamide, a pharmacologically acceptable salt thereof, or a solvate of said compound or said salt.

32. The method according to claim 15, wherein the VEGF receptor kinase inhibitor is a methanesulfonic acid salt of 4-(3-chloro-4-(cyclopropylaminocarbonyl)aminophenoxy)-7-methoxy-6-quinolinecarboxamide.

33. The method according to claim 15, wherein the VEGF receptor kinase inhibitor is a compound represented by the following general formula (III), a pharmacologically acceptable salt thereof, or a solvate of said compound or said salt: 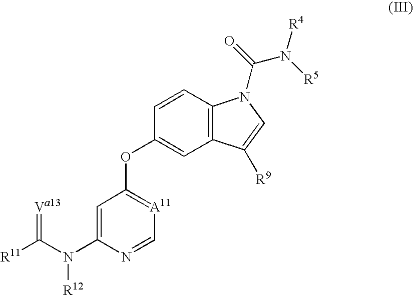
wherein R¹¹ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s), a 3- to 10-membered non-aromatic heterocyclic group which may have a substituent(s) or a mono-C₁₋₆ alkylamino group which may have a substituent(s);
R¹² a hydrogen atom or a C₁₋₆ alkyl group which may have a substituent(s);
V^{a13} is a hydrogen atom or a sulfur atom;
A¹¹ is a carbon atom or a nitrogen atom which may have a substituent(s);
R⁴ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₂₋₇ acyl group which may have a substituent(s) or a C₂₋₇ alkoxycarbonyl group which may have a substituent(s);
R⁵ is a hydrogen atom, a C₁₋₆ alkyl group which may have a substituent(s), a C₂₋₆ alkenyl group which may have a substituent(s), a C₂₋₆ alkynyl group which may have a substituent(s), a C₃₋₈ cycloalkyl group which may have a substituent(s), a C₆₋₁₀ aryl group which may have a substituent(s), a 5- to 10-membered heteroaryl group which may have a substituent(s) or a 3- to 10-membered non-aromatic heterocyclic group which may have a substituent(s); and
R⁹ is a hydrogen atom, a halogen atom or a C₁₋₆ alkyl group which may have a substituent(s).

34. The method according to claim 33, wherein R¹¹ is a 3- to 10-membered non-aromatic heterocyclic group which may have a substituent(s) or a mono-C₁₋₆ alkylamino group which may have a substituent(s).

35. The method according to claim 33, wherein R¹¹ is any one group selected from the groups represented by the following formulas: 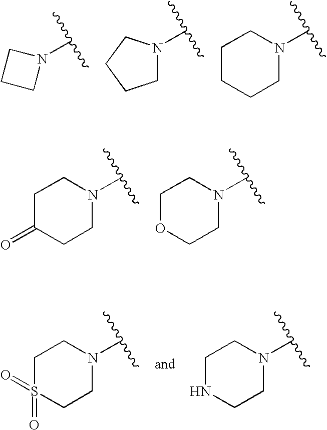
wherein said group may have at least one substituent selected from the group of substituents consisting of hydroxyl group, C₁₋₆ alkyl, C₃₋₈ cycloalkyl and groups represented by the formulas: 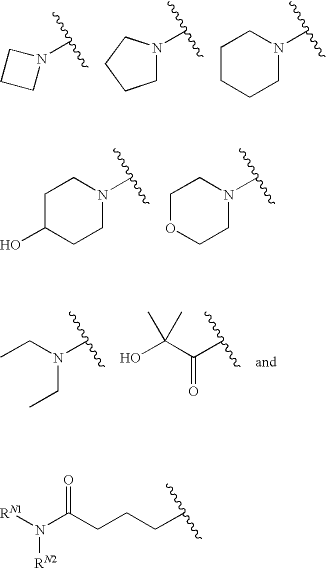
wherein R^{N1} and R^{N2} independently of each other represent a hydrogen atom or a C₁₋₆ alkyl group which may have a substituent(s).

36. The method according to claim 33, wherein R¹¹ is any one group selected from the group consisting of groups represented by the following formulas:

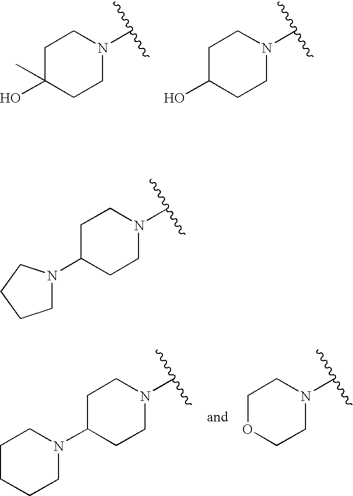

37. The method according to claim 33, wherein R¹² is a hydrogen atom.

38. The method according to claim 33, wherein V^{a13} is an oxygen atom.

39. The method according to claim 33, wherein A¹¹ is a carbon atom.

40. The method according to claim 33, wherein R⁴ is a hydrogen atom.

41. The method according to claim 33, wherein R⁵ is a C₁₋₆ alkyl group or a C₃₋₈ cycloalkyl group.

42. The method according to claim 33, wherein R⁵ is a methyl group.

43. The method according to claim 33, wherein R⁹ is a hydrogen atom.

44. The method according to claim 15, wherein the VEGF receptor kinase inhibitor is at least one compound selected from the group consisting of:
5-(2-(((4-hydroxy-4-methylpiperidine-1-yl)carbonyl)amino)pyridine-4-yloxy)-1H-indole-1-carboxylic acid methylamide,
N1-methyl-5-(2-((4-hydroxypiperidino)carbonyl)amino-4-pyridyl)oxy-1H-1-indolecarboxamide,
N1-methyl-5-(2-(((4-pyrrolizine-1-yl)piperidine-1-yl)carbonyl)amino)pyridine-4-yloxy)-1H-1-indolecarboxamide,
N1-methyl-5-(2-(((4-piperidine-1-yl)piperidine-1-yl)carbonyl)amino)pyridine-4-yloxy)-1H-1-indolecarboxamide, and
N4-(4-(1-(methylamino)carbonyl-1H-5-indolyl)oxy-2-pyridyl)-4-morpholinecarboxamide;
or a pharmacologically acceptable salt of said compound, or a solvate of said compound or said salt.

45. The method according to claim 15, wherein the VEGF receptor kinase inhibitor is at least one compound selected from the group consisting of:
(1) N-(4-bromo-2-fluorophenyl)-6-methoxy-7-[2-(1H-1,2,3-triazole-1-yl)-ethoxy]quinazoline-4-amine
(2) N-(4-bromo-2-fluorophenyl)-6-methoxy-7-[(1-methylpiperidine-4-yl)-methoxy]quinazoline-4-amine
(3) 3-[(2,4-dimethylpyrrol-5-yl)methylene]-2-indolinone
(4) (Z)-3-[(2,4-dimethyl-5-(2-oxo-1,2-dihydroindole-3-ylidenemethyl)-1H-pyrrole-3-yl)-propionic acid
(5) 5-(5-fluoro-2-oxo-1,2-dihydroindole-3-ylidenemethyl)-2,4-dimethyl-1H-pyrrole-3-carboxylic acid (2-diethylaminoethyl)amide
(6) N,N-dimethylglycine-3-{5,6,7,13-tetrahydro-9-[(1-methylethoxy)methyl]-5-oxo-12H-indeno(2,1-a)pyrrolo[3,4-c]carbazole-12-yl}propylester
(7) 3-(4-bromo-2,6-difluoro-benzyloxy)-5-[3-(4-pyrrolizine-1-yl-butyl)-ureido]-isothiazole-4-carboxylic acid amide
(8) N-{2-chloro-4-[(6,7-dimethoxy-4-quinazolinyl)oxy]phenyl}-N′-propylurea
(9) 1-(4-chloroanilino)-4-(4-pyridylmethyl)phthalazine
(10) N-{2-chloro-4-[(6,7-dimethoxy-4-quinolyl)oxy]phenyl}-N′-[5-methyl-3-isoxazolyl)urea
(11) 4-[(4-fluoro-2-methylindole-5-yl)oxy]-6-methoxy-7-[3-(pyrrolizine-1-yl)-propoxy]quinazoline
(12) 6-[2-(methylcarbamoyl)phenylsulphanyl]-3-E-[2-(pyridine-2-yl)-ethenyl]indazole
(13) 5-((Z)-(5-fluoro-2-oxo-1,2-dihydro-3H-indole-3-ylidene)methyl)-N-((2S)-2-hydroxy-3-morpholine-4-ylpropyl)-2,4-dimethyl-1H-pyrrole-3-carboxamide
(14) 3-((quinoline-4-ylmethyl)amino)-N-(4-(trifluoromethoxy)phenyl)thiophene-2-carboxamide
(15) 6-(2,6-dichlorophenyl)-8-methyl-2-phenylamino-8H-pyrido[2,3-d]-pyrimidine-7-one
(16) 2-((1,6-dihydro-6-oxo-pyridine-3-ylmethyl)amino)-N-(3-(trifluoromethyl)-phenyl)-3-pyridine-carboxamide
(17) 4-(4-(4-chloro-phenylamino)-furo[2,3-d]pyridazine-7-yloxymethyl)-pyridine-2-carboxylic acid methylamide
(18) N-(3-trifluoromethyl-4-chlorophenyl)-N′-(4-(2-methylcarbamoylpyridine-4-yl)oxyphenyl)urea
(19) 4-amino-5-fluoro-3-(6-(4-methyl-piperazine-1-yl)-1H-benzimidazole-2-yl)-1H-quinoline-2-one
(20) 4-(4-(1-amino-1-methyl-ethyl)-phenyl)-2-(4-(2-morpholine-4-yl-ethyl)-phenylamino)-pyrimidine-5-carbonitrile
(21) [6-[4-[(4-ethylpiperazine-1-yl)methyl]phenyl]-7H-pyrrolo[2,3-d]pyrimidine-4-yl]-((R)-1-phenylethyl)amine
(22) 9-(1-methylethoxy)methyl-12-(3-hydroxypropyl)-6H,7H,13H-indeno[2,1-a]-pyrrole[3,4-c]carbazole-5-one
(23) N-(2,4-difluorophenyl)-N′-{4-[(6,7-dimethoxy-4-quinolyl)-oxy]-2-fluorophenyl}urea
(24) N-[4-(3-amino-1H-indazole-4-yl)phenyl]-N′-(2-fluoro-5-methylphenyl)urea
(25) 2-methyl-6-[2-(1-methyl-1H-imidazole-2-yl)-thieno[3,2-b]pyridine-7-yloxy]-benzo[b]thiophene-3-carboxylic acid methylamide
(26) (R)-1-(4-(4-fluoro-2-methyl-1H-indole-5-yloxy)-5-methylpyrrolo[1,2-f]-[1,2,4]triazine-6-yloxy)propane-2-ol
(27) (S)-((R)-1-(4-(4-fluoro-2-methyl-1H-indole-5-yloxy)-5-methylpyrrolo[1,2-f][1,2,4]triazine-6-yloxy)propane-2-ol)-2-aminopropanoate
(28) 3-[(4-morpholine-4-yl-phenylamino)-methylene]-1,3-dihydroindole-2-one
(29) 5-[[4-(2,3-dimethyl-2H-indazole-6-yl)methylamino]pyrimidine-2-yl]amino]-2-methylbenzenesulfonamide
(30) (3Z)-3-[6-(2-morpholine-4-ylethoxy)quinoline-2(1H)-ylidene]-1,3-dihydro-2H-indole-2-one, and
(31) 2-((2-((4-(4-(4-(tert-butyl)anilino)phenoxy)-6-methoxy-7-quinolyl)oxy)ethyl)amino)-1-ethanol;
or a pharmacologically acceptable salt of said compound, or a solvate of said compound or said salt.

46. The method according to claim 1, wherein the angiogenesis inhibitor is an anti-VEGF receptor antibody.

47. The method according to claim 46, wherein the anti-VEGF receptor antibody is at least one antibody selected from the group consisting of 2C3 antibody, IMC-1121b, IMC-18F1, IMC-1C11 and IMC-2C6.

48. The method according to claim 1, wherein the angiogenesis inhibitor is an anti-VEGF antibody.

49. The method according to claim 48, wherein the anti-VEGF antibody is bevacizumab.

50. The method according to claim 1, wherein the angiogenesis inhibitor is at least one agent selected from the group consisting of PI88, AVE-0005, EG-3306, RPI-4610, NM-3, VGA-1155, VEGF trap and pegaptanib sodium.

51. The method according to claim 1, wherein the angiogenesis inhibitor is at least one agent selected from the group consisting of FGF receptor kinase inhibitor, PDGF receptor kinase inhibitor, EGF receptor kinase inhibitor, anti-FGF receptor antibody, anti-PDGF receptor antibody, anti-EGF receptor antibody, anti-FGF antibody, anti-PDGF antibody and anti-EGF antibody.

52. The method according to claim 51, wherein the FGF receptor kinase inhibitor is at least one compound selected from the group consisting of:
(1) 1-[2-amino-6-(3,5-dimethoxyphenyl)-pyrido(2,3-d)pyrimidine-7-yl]-3-tert-butylurea
(2) 1-tert-butyl-3-[2-(4-diethylamino)butylamino-6-(3,5-dimethoxyphenyl)-pyrido(2,3-d)pyrimidine-7-yl]urea
(3) (S)-((R)-1-(4-(4-fluoro-2-methyl-1H-indole-5-yloxy)-5-methylpyrrolo[1,2-f]-[1,2,4]triazine-6-yloxy)propane-2-ol)-2-aminopropanoate
(4) 4-[4-[N-(4-nitrophenyl)carbamoyl]-1-piperazinyl]-6,7-dimethoxyquinazoline
(5) 4-amino-5-fluoro-3-(6-(4-methyl-piperazine-1-yl)-1H-benzimidazole-2-yl)-1H-quinoline-2-one
(6) 2-((2-((4-(4-(4-(tert-butyl)anilino)phenoxy)-6-methoxy-7-quinolyl)oxy)ethyl)-amino)-1-ethanol, and
(7) (Z)-3-[(2,4-dimethyl-5-(2-oxo-1,2-dihydroindole-3-ylidenemethyl)-1H-pyrrole-3-yl)-propionic acid;
or a pharmacologically acceptable salt of said compound, or a solvate of said compound or said salt.

53. The method according to claim 51, wherein the PDGF receptor kinase inhibitor is at least one compound selected from the group consisting of:
(1) 4-(4-methylpiperazine-1-ylmethyl)-N-[4-methyl-3-[4-(3-pyridyl)pyrimidine-2-ylamino]phenyl]benzeneamide
(2) 6-[2-(methylcarbamoyl)phenylsulphanyl]-3-E-[2-(pyridine-2-yl)ethenyl]-indazole
(3) 1-{2-[5-(2-methoxy-ethoxy)-benzoimidazole-1-yl]-quinoline-8-yl}-piperidine-4-ylamine
(4) 4-[4-[N-(4-nitrophenyl)carbamoyl]-1-piperazinyl]-6,7-dimethoxyquinazoline
(5) 4-amino-5-fluoro-3-(6-(4-methyl-piperazine-1-yl)-1H-benzimidazole-2-yl)-1H-quinoline-2-one
(6) (4-tert-butylphenyl){4-[(6,7-dimethoxy-4-quinolyl)oxy]phenyl}methaneone
(7) 5-methyl-N-[4-(trifluoromethyl)phenyl]-4-isoxazolecarboxamide
(8) trans-4-[(6,7-dimethoxyquinoxaline-2-yl)amino]cyclohexanol
(9) (Z)-3-[(2,4-dimethyl-5-(2-oxo-1,2-dihydroindole-3-ylidenemethyl)-1H-pyrrole-3-yl)-propionic acid
(10) 5-(5-fluoro-2-oxo-1,2-dihydroindole-3-ylidenemethyl)-2,4-dimethyl-1H-pyrrole-3-carboxylic acid (2-diethylaminoethyl)amide
(11) 1-(4-chloroanilino)-4-(4-pyridylmethyl)phthalazine, and
(12) N-[4-(3-amino-1H-indazole-4-yl)phenyl-N′-(2-fluoro-5-methylphenyl)urea;
or a pharmacologically acceptable salt of said compound, or a solvate of said compound or said salt.

54. The method according to claim 51, wherein the EGF receptor kinase inhibitor is at least one compound selected from the group consisting of:
(1) 4-(3-chloro-4-fluorophenylamino)-7-methoxy-6-(3-(4-morpholino)propoxy-quinazoline)
(2) 4-(3-ethynylphenylamino)-6,7-bis(2-methoxyethoxy)-quinazoline
(3) N-[3-chloro-4-[(3-fluorobenzyl)oxy]phenyl]-6-[5-[[[2-(methylsulfonyl)ethyl]-amino]methyl]furan-2-yl]quinazoline-4-amine
(4) N-[4-[N-(3-chloro-4-fluorophenyl)amino]-7-[3-(4-morpholinyl)propoxy]-quinazoline-6-yl]acrylamide
(5) (2E)-N-[4-[(3-chloro-4-fluorophenyl)amino]-3-cyano-7-ethoxy-6-quinolinyl]-4-(dimethylamino)-2-butenamide
(6) [6-[4-[(4-ethylpiperazine-1-yl)methyl]phenyl]-7H-pyrrolo[2,3-d]pyrimidine-4-yl]-((R)-1-phenylethyl)amine, and
(7) (E)-N-{-4-[3-chloro-4-(2-pyridinylmethoxy)anilino]-3-cyano-7-ethoxy-6-quinolinyl}-4-(dimethylamino)-2-butenamide;
or a pharmacologically acceptable salt of said compound, or a solvate of said compound or said salt.

55. The method according to claim 51, wherein the anti-EGF receptor antibody is at least one antibody selected from the group consisting of cetuximab, panitumumab, matuzumab, nimotuzumab, IMC-11F8 and MDX-447.

56. A kit for use in the method according to claim 1, comprising at least one antibody selected from the group consisting of anti-α-SMA antibody, anti-desmin antibody, anti-chondroitin sulfate proteoglycan 4 antibody, anti-calponin antibody, anti-caldesmon antibody and anti-PDGF receptor antibody.

57. A kit for use in the method according to claim 1, comprising anti-α-SMA antibody.

58. A kit for use in the method according to claim 1, comprising a polynucleotide comprising a sequence complementary to at least a part of a transcript RNA from at least one gene selected from the group consisting of α-SMA gene, desmin gene, chondroitin sulfate proteoglycan 4 gene, calponin gene, caldesmon gene and PDGF receptor gene.

59. A kit for use in the method according to claim 1, comprising a polynucleotide comprising a sequence complementary to at least a part of a transcript RNA from desmin gene.
